(19) United States
(12) Patent Application Publication
(10) Pub. No.: US 2010/0104584 A1
(43) Pub. Date: Apr. 29, 2010

(54) GENES INVOLVED IN MITOCHONDRIAL BIOGENESIS

(76) Inventors: Jian Chen, Cambridge, MA (US); Dan Garza, Cambridge, MA (US); Hao Li, Cambridge, CA (US); Susan C. Stevenson, Cambridge, MA (US); Zhidan Wu, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/526,674

(22) PCT Filed: Feb. 14, 2008
(86) PCT No.: PCT/EP08/51814

§ 371 Date: Aug. 11, 2009

Related U.S. Application Data
(60) Provisional application No. 60/890,351, filed on Feb. 16, 2007.

(51) Int. Cl. A61K 39/395; A61P 3/10; A61K 31/7052; C12Q 1/68; G01N 33/68; A61P 35/00

(52) U.S. Cl. 424/172.1; 514/44 A; 514/44 R; 435/6; 436/86

(57) ABSTRACT

The invention discloses suitable gene and polypeptide targets for the development of new therapeutics to treat, prevent or ameliorate conditions associated with mitochondrial dysfunction. The invention also relates to methods to treat, prevent or ameliorate said conditions and pharmaceutical compositions therefor, as well as to a method to identify compounds with therapeutic usefulness to treat conditions associated with mitochondrial dysfunction.

### BACKGROUND OF THE INVENTION

Mitochondria are cytoplasmic organelles that not only are involved in ATP production, but which also contribute to thermogenesis, free radial production, calcium homeostasis and apoptosis.

Mitochondrial dysfunction is associated with a wide range of human disorders and conditions, including neurodegenerative diseases (Orth M, et al. (2001) Amer. Journal of Med. Gen. 106 (1):27), cardiovascular disease (Ballinger C A (2005) Free Radical Biol. & Med. 38:1278), diabetes (Lowell B B, et al. (2005) Science 307(5708):384), aging (Dufour E, et al. (2004) Biochimica et Biophysica Acta 1658(1-2):122) and cancer (Wallace D C (2005) Annual Review of Gen. 39:359). Mitochondria-targeted therapy has in many instances been suggested for these human diseases (Wallace 2005; McLeod C J, et al. (2005) Trends in CV Med. 15 (3):118) (Schapira A H (2006) Lancet 368(9529):70) (Manczak M, et al. (2006) Human Mol. Gen. 15(9):1437) (Armstrong J S et al. (2006) Bioessays 28(3):253).

To better understand the molecular mechanisms regulating mitochondrial biogenesis and to identify new regulators of mitochondrial function and biological activity, Applicants performed both in vitro and in vivo assays, including an unbiased whole-genome RNAi screen in Drosophila S2 cells.

Described herein is the identification of various biological processes and signaling pathways in regulating mitochondrial function. Also provided herein is an analysis of modulators of mitochondrial function, as identified at least in part by a whole genome scale RNAi screen. Applicants have discovered a variety of new genes involved in mitochondrial biogenesis, and which are also implicated in conditions associated with mitochondrial dysfunction. It is contemplated herein that these genes and the proteins encoded thereby may serve as drug targets for the development of therapeutics to treat, prevent or ameliorate conditions associated with mitochondrial dysfunction, e.g., neurodegenerative diseases, cardiovascular disease, diabetes, age-related disorders, and cancers.

### SUMMARY OF THE INVENTION

The instant application discloses human orthologs of several Drosophila genes as suitable targets for the development of new therapeutics to treat, prevent or ameliorate conditions associated with mitochondrial dysfunction including, but not limited to, neurodegenerative diseases (e.g., Parkinson's Disease, Alzheimer's Disease, Huntington's Disease), cardiovascular diseases, diabetes, age-related disorders, and cancers. Thus, in one aspect the invention relates to a method to identify modulators useful to treat, prevent or ameliorate said conditions comprising:

(a) assaying for the ability of a candidate modulator, in vitro or in vivo, to modulate a biological activity of a protein selected from the group consisting of the proteins disclosed in TABLE I and/or modulate the expression of a gene encoding said protein; and which can further include

(b) assaying for the ability of an identified modulator to modulate mitochondrial citrate synthase activity in animal models and/or in clinical studies with subjects with said conditions.

In another aspect, the invention relates to a method to treat, prevent or ameliorate conditions associated with mitochondrial dysfunction, comprising administering to a subject in need thereof an effective amount of a modulator of a protein selected from the group consisting of the proteins disclosed in TABLE I, wherein said modulator, e.g., inhibits or enhances a biological activity of said protein. In one aspect, the modulator comprises antibodies to said protein or fragments thereof, wherein said antibodies can inhibit a biological activity of said protein in said subject.

In another aspect, the modulator inhibits or enhances the RNA expression of a gene encoding for a protein selected from the group consisting of the proteins disclosed in TABLE I. In a further aspect, the modulator comprises any one or more substances selected from the group consisting of antisense oligonucleotides, triple-helix DNA, ribozymes, RNA and DNA aptamers, siRNA and double- or single-stranded RNA, wherein said substances are designed to inhibit RNA expression of gene encoding said protein.

In another aspect, the invention relates to a method to treat, prevent or conditions associated with mitochondrial dysfunction, comprising administering to a subject in need thereof a pharmaceutical composition comprising an effective amount of a modulator of a protein selected from the group consisting of the proteins disclosed in TABLE I. In various aspects, said pharmaceutical composition comprises antibodies to said protein or fragments thereof, wherein said antibodies can inhibit a biological activity of said protein in said subject and/or any one or more substances selected from the group consisting of antisense oligonucleotides, triple-helix DNA, ribozymes, RNA and DNA aptamers, siRNA and double- or single-stranded RNA, wherein said substances are designed to inhibit RNA expression of gene encoding said protein. It is contemplated herein that one or more modulators of one or more of said proteins may be administered concurrently.

In another aspect, the invention relates to a pharmaceutical composition comprising a modulator to a protein selected from the group consisting of the proteins disclosed in TABLE I in an amount effective to treat, prevent or ameliorate a condition associated with mitochondrial dysfunction, in a subject in need thereof. In one aspect, said modulator may, e.g., inhibit or enhance a biological activity of said protein. In a further aspect, said modulator comprises antibodies to said protein or fragments thereof, wherein said antibodies can, e.g., inhibit a biological activity of said protein.

In a further aspect, said pharmaceutical composition comprises a modulator which may, e.g., inhibit or enhance RNA expression of gene encoding said protein. In a further aspect, said modulator comprises any one or more substances selected from the group consisting of antisense oligonucleotides, triple-helix DNA, ribozymes, RNA or DNA aptamers, siRNA or double- or single-stranded RNA directed to a nucleic acid sequence of said protein, wherein said substances are designed to inhibit RNA expression of gene encoding said protein.

In another aspect, the invention relates to a method to diagnose subjects suffering from a condition associated with mitochondrial dysfunction who may be suitable candidates for treatment with modulators to a protein selected from the group consisting of the proteins disclosed in TABLE I, comprising detecting levels of any one or more of said proteins in a biological sample from said subject wherein subjects with altered levels compared to controls would be suitable candidates for modulator treatment.

In another aspect, the invention relates to a method to diagnose subjects suffering from a condition associated with mitochondrial dysfunction, who may be suitable candidates for treatment with modulators to a protein selected from the group consisting of the proteins disclosed in TABLE I, comprising assaying messenger RNA (mRNA) levels of any one or more of said protein in a biological sample from said subject, wherein subjects with altered levels compared to controls would be suitable candidates for modulator treatment.

In yet another aspect, there is provided a method to treat, prevent or ameliorate conditions associated with mitochondrial dysfunction, comprising:

(a) assaying for mRNA and/or protein levels of a protein selected from the group consisting of the proteins disclosed in TABLE I in a subject; and

(b) administering to a subject with altered levels of mRNA and/or protein levels compared to controls a modulator to said protein in an amount sufficient to treat, prevent or ameliorate said condition.

In particular aspects, said modulator inhibits or enhances a biological activity of said protein or RNA expression of gene encoding said protein.

In yet another aspect of the present invention, there are provided assay methods and diagnostic kits comprising:

(a) the components necessary to detect mRNA levels or protein levels of any one or more proteins selected from the group consisting of the proteins disclosed in TABLE I in a biological sample, said kit comprising, e.g., polynucleotides encoding any one or more proteins selected from the group consisting of the proteins disclosed in TABLE I; and

(b) nucleotide sequences complementary to said protein;

(c) any one or more of said proteins, or fragments thereof of antibodies that bind to any one or more of said proteins, or to fragments thereof.

In a preferred aspect, such kits also comprise instructions detailing the procedures by which the kit components are to be used.

The present invention also pertains to the use of a modulator to a protein selected from the group consisting of the proteins disclosed in TABLE I, in the manufacture of a medicament for the treatment, prevention or amelioration of conditions associated with mitochondrial dysfunction. In one aspect, said modulator comprises any one or more substances selected from the group consisting of antisense oligonucleotides, triple-helix DNA, ribozymes, RNA aptamer, siRNA and double- or single-stranded RNA, wherein said substances are designed to inhibit gene expression of said protein. In yet a further aspect, said modulator comprises one or more antibodies to said protein or fragments thereof, wherein said antibodies or fragments thereof can, e.g., inhibit a biological activity of said protein.

The invention also pertains to a modulator to a protein selected from the group consisting of the proteins disclosed in TABLE I for use as a pharmaceutical. In one aspect, said modulator comprises any one or more substances selected from the group consisting of antisense oligonucleotides, triple-helix DNA, ribozymes, RNA aptamer, siRNA and double- or single-stranded RNA, wherein said substances are designed to inhibit gene expression of said protein. In yet a further aspect, said modulator comprises one or more antibodies to said protein or fragments thereof, wherein said antibodies or fragments thereof can, e.g., inhibit a biological activity of said protein.

Other objects, features, advantages and aspects of the present invention will become apparent to those of skill from the following description. It should be understood, however, that the following description and the specific examples, while indicating preferred aspects of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE FIGURES

FIG. 1. Development of CS activity assay for genome wide RNAi screen. (a) Dosage response of CS activity after CS RNAi (n=48). CS activities (Vmax/Ren) were reduced by RNAi with a dosage dependent manner. Seven dosages of CS dsRNA were used, from CS1 to CS7 were 0.5, 1, 2, 4, 8, 16, 32 ug/ml/10⁶ cells. (b) Stability of CS activity in S2 cells. CS activity was calculated as the Vmax rate, the change of absorbance over time (Vmax=dA/dt). Optical density (OD) at 412 nm for cell lysates from LacZ RNAi and CS RNAi was shown on y-axis. X-axis shows time in seconds, data were captured every 46 seconds. (c) Linear range of CS activity in S2 cells (n=48). The y-axis shows CS activity (Vmax/ug) normalized by total protein concentration. The x-axis shows the total protein concentrations (ug) of cell lysates. The protein concentration in ug labeled next to each data points. Log scales are applied to both x and y-axis. (d) CS activity (Vmax/Ren) after RNAi with selected control genes (n=48). Value for CS is 0.22. p value is labeled next to the column. “*” indicates p<0.05, “*” indicates p<0.01. Error bars in a and d indicate the standard errors.

FIG. 2. Whole genome RNAi screen. (a) Primary screen. Spotfire scatter plot shows NZ of CS activity (Vmax/Ren) for primary screen. X and y-axis represent 1D normalized NZ for replica b and replica a, respectively, log scale applied. Each spot represents each dsRNA. (b) Confirmation screen for the primary hits. Spotfire scatter plot shows that hits were selected by their p-values against LacZ controls. Hits that negatively affect CS activities (their RNAi result in increasing citrate synthase activity) were selected if their p<0.05. Hits that positively affect CS activities (their RNAi result in reducing CS activity) were selected if their p<0.01. The x-axis represents average fold of CS activities of six replicates against the LacZ controls. The y-axis represents p-value of the hits. (c) Distribution of LacZ RNAi controls. LacZ RNAi controls in the confirmation screen were shown. (d) Classification of the confirmed hits by their molecular functions. (e) Classification of the confirmed hits by their biological processes and pathways.

FIG. 3. HDAC1 and HDAC6 modulate mitochondrial functions in vivo. (a) CS activity in S2 cells treated with HADC1 and HDAC6 RNAi (n=6). LacZ RNAi acts as negative control and CS RNAi as positive control. P-value were calculated compared to LacZ group. (b) HDAC1 protein was highly reduced in transgenic HDAC1 RNAi fly as shown by western blot. Lysates from HDAC1 RNAi and control siblings were blotted with antibody against HDAC1 and tubulin. Tubulin levels were used as loading controls. (c) HDAC6 protein was highly reduced in transgenic HDAC6 RNAi fly as shown by western blot. Lysates from transgenic HDAC6 RNAi and control siblings were blotted with antibodies against HDAC6 and tubulin. Tubulin levels were used as loading controls. (d) CS activity in transgenic HDAC1 RNAi flies and control siblings (n=8). (e) CS activity in transgenic HDAC6 RNAi flies and control siblings (n=8). (f) COX activity in transgenic HDAC1 RNAi flies and control siblings (n=8). (g) COX activity in transgenic HDAC6 RNAi flies and control siblings (n=8). “*” indicates p<0.05, “*” indicates p<0.01, “***” indicates p<0.001. Error bars indicate the standard errors.

FIG. 4. CS activity in heterozygous mutants. (a) CS, (b) CG3249, (c) vimar, (d) Src42A, (e) Src42A, (1) klumpfuss, (g) smt3, (h) smt3, (i) barren, (j) barren. n=8 for each experiment. “*” indicates p<0.05, “**” indicates p<0.01. Error bars indicate the standard errors.

### DETAILED DESCRIPTION OF THE INVENTION

All patent applications, patents and literature references cited herein are hereby incorporated by reference in their entirety.

In practicing the present invention, many conventional techniques in molecular biology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Edition, Cold Spring Harbor Laboratory Press, Cold Spring Harbor, N.Y. (1989); DNA Cloning: A Practical Approach, Vols. I and II, Glover, Ed. (1985); Oligonucleotide Synthesis, Gait, Ed. (1984); Nucleic Acid Hybridization, Hames and Higgins, Eds. (1985); Transcription and Translation, Hames and Higgins, Eds. (1984); Animal Cell Culture, Freshney, Ed. (1986); Immobilized Cells and Enzymes, IRL Press (1986); Perbal, A Practical Guide to Molecular Cloning; the series, Meth Enzymol, Academic Press, Inc. (1984); Gene Transfer Vectors for Mammalian Cells, Miller and Calos, Eds., Cold Spring Harbor Laboratory Press, NY (1987); and Methods in Enzymology, Vols. 154 and 155, Wu and Grossman, and Wu, Eds., respectively (1987). Well-known Drosophila-molecular genetics techniques can be found, e.g., in Drosophila, A Practical Approach, Robert, Ed., IRL Press, Washington D.C. (1986).

Descriptions of flystocks can be found in the Flybase database at http://flybase.bio.indiana.edu.

As used herein and in the appended claims, the singular forms “a”, “an” and “the” include plural reference unless the context clearly dictates otherwise. Thus, e.g., reference to “the antibody” is a reference to one or more antibodies and equivalents thereof known to those skilled in the art, and so forth.

“Nucleic acid sequence”, as used herein, refers to an oligonucleotide, nucleotide or polynucleotide, and fragments or portions thereof, and to DNA or RNA of genomic or synthetic origin that may be single- or double-stranded, and represent the sense or antisense strand.

The term “degenerate nucleotide sequence” refers to a sequence of nucleotides that includes one or more degenerate codons (as compared to a reference polynucleotide molecule that encodes a polypeptide). Degenerate codons contain different triplets of nucleotides, but encode the same amino acid residue, i.e., GAU and GAC triplets each encode Asp. Some polynucleotides encompassed by a degenerate sequence may have some variant amino acids, but one of ordinary skill in the art can easily identify such variant sequences by reference to the amino acid sequences encoding the proteins disclosed in TABLE I. Variants of the proteins disclosed in TABLE I can be generated through DNA shuffling as disclosed by Stemmer, Nature, Vol. 370, No. 6488, 389-391 (1994); and Stemmer, Proc Natl Acad Sci USA, Vol. 91, No. 22, 10747-10751 (1994). Variant sequences can be readily tested for functionality as described herein.

“Allelic variant” refers to any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

Allelic variants can be cloned by probing cDNA or genomic libraries from different individuals according to standard procedures. Allelic variants of the DNA sequences encoding proteins disclosed in TABLE I and variants thereof, including those containing silent mutations and those in which mutations result in amino acid sequence changes, are within the scope of the present invention.

“Splice variant” refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term “splice variant” is also used herein to denote a protein encoded by a splice variant of an mRNA transcribed from a gene.

The term “antisense,” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. The designation “negative” is sometimes used in reference to the antisense strand, and “positive” is sometimes used in reference to the sense strand.

“cDNA” refers to DNA that is complementary to a portion of mRNA sequence and is generally synthesized from an mRNA preparation using reverse transcriptase.

As contemplated herein, antisense oligonucleotides, triple-helix DNA, RNA aptamers, ribozymes, siRNA and double- or single-stranded RNA are directed to a nucleic acid sequence such that the nucleotide sequence chosen will produce gene-specific inhibition of gene expression. For example, knowledge of a nucleotide sequence may be used to design an antisense molecule which gives strongest hybridization to the mRNA. Similarly, ribozymes can be synthesized to recognize specific nucleotide sequences of a gene and cleave it. See Cech, JAMA, Vol. 260, No. 20, 3030-3034 (1988). Techniques for the design of such molecules for use in targeted inhibition of gene expression is well-known to one of skill in the art.

The individual proteins/polypeptides referred to herein include any and all forms of these proteins including, but not limited to, partial forms, isoforms, variants, precursor forms, the full-length protein, fusion proteins containing the sequence or fragments of any of the above, from human or any other species. Protein homologs or orthologs which would be apparent to one of skill in the art are included in this definition. These proteins/polypeptides may further comprise variants wherein the resulting polypeptide will be at least 80-90% or in other aspects, at least 95%, 96%, 97%, 98% or 99% identical to the corresponding region of a sequence selected from TABLE I. Percent sequence identity is determined by conventional methods. See, e.g., Altschul and Erickson, Bull Math Biol, Vol. 48, Nos. 5-6, 603-616 (1986); and Henikoff and Henikoff, Proc Natl Acad Sci USA, Vol. 89, No. 22, 10915-10919 (1992). Briefly, two amino acid sequences are aligned to optimize the alignment scores using a gap opening penalty of 10, a gap extension penalty of 1, and the “BLOSUM62” scoring matrix of Henikoff and Henikoff. The percent identity is then calculated as:

(total number of identical matches)/(length of the longer sequence plus the number of gaps introduced into the longer sequence in order to align the two sequences)×100

It is also contemplated that the terms proteins or polypeptides refer to proteins isolated from naturally-occurring sources of any species, such as genomic DNA libraries, as well as genetically-engineered host cells comprising expression systems, or produced by chemical synthesis using, for instance, automated peptide synthesizers or a combination of such methods. Means for isolating and preparing such polypeptides are well-understood in the art.

The term “sample,” as used herein, is used in its broadest sense. A biological sample from a subject may comprise blood, urine, brain tissue, primary cell lines, immortalized cell lines or other biological material with which protein activity or gene expression may be assayed. A biological sample may include, e.g., blood, tumors or other specimens from which total RNA may be purified for gene expression profiling using, e.g., conventional glass chip microarray technologies, such as Affymetrix chips, RT-PCR or other conventional methods.

As used herein, the term “antibody” refers to intact molecules, as well as fragments thereof, such as Fa, F(ab′)₂ and Fv, which are capable of binding the epitopic determinant. Antibodies that bind specific polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein. Commonly used carriers that are chemically coupled to peptides include bovine serum albumin and thyroglobulin. The coupled peptide is then used to immunize an animal, e.g., a mouse, goat, chicken, rat or a rabbit.

The term “humanized antibody,” as used herein, refers to antibody molecules in which amino acids have been replaced in the non-antigen binding regions in order to more closely resemble a human antibody, while still retaining the original binding ability.

“TABLE I,” as used herein, means the following:

TABLE I

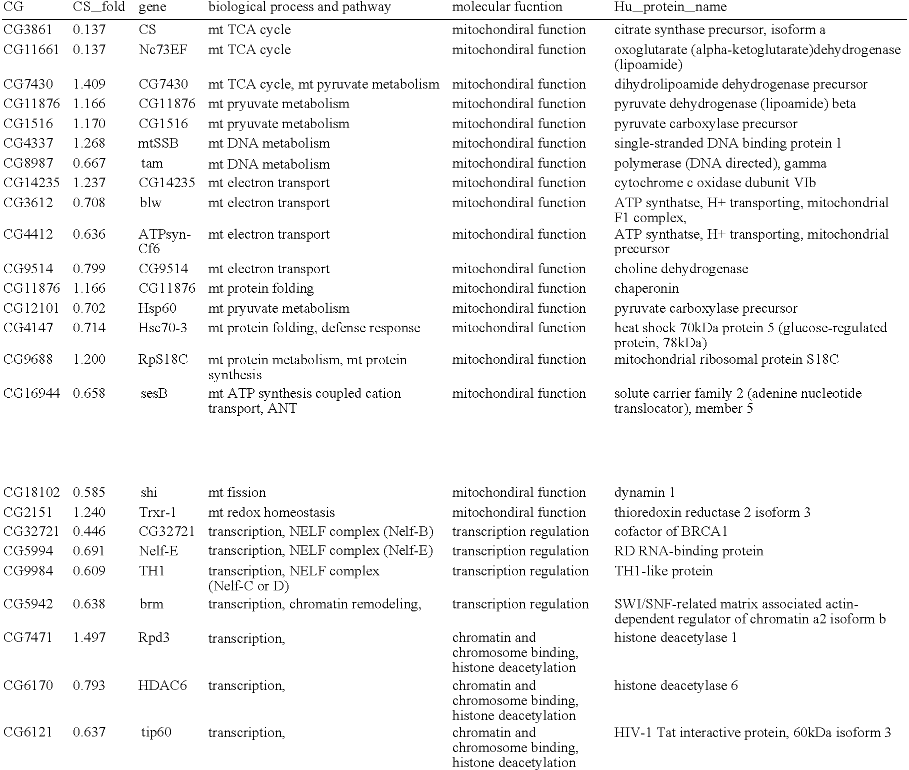

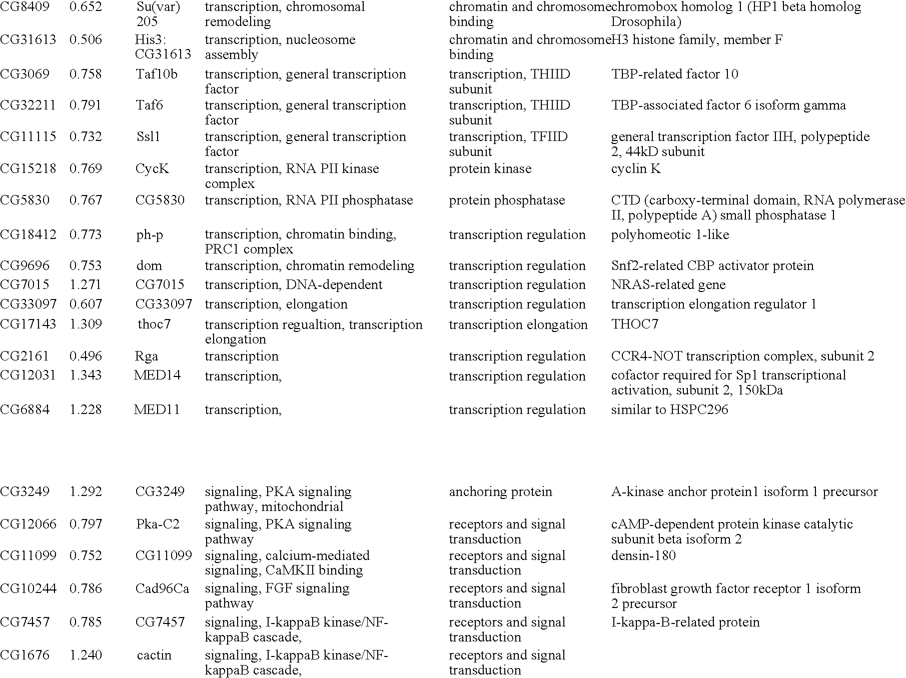

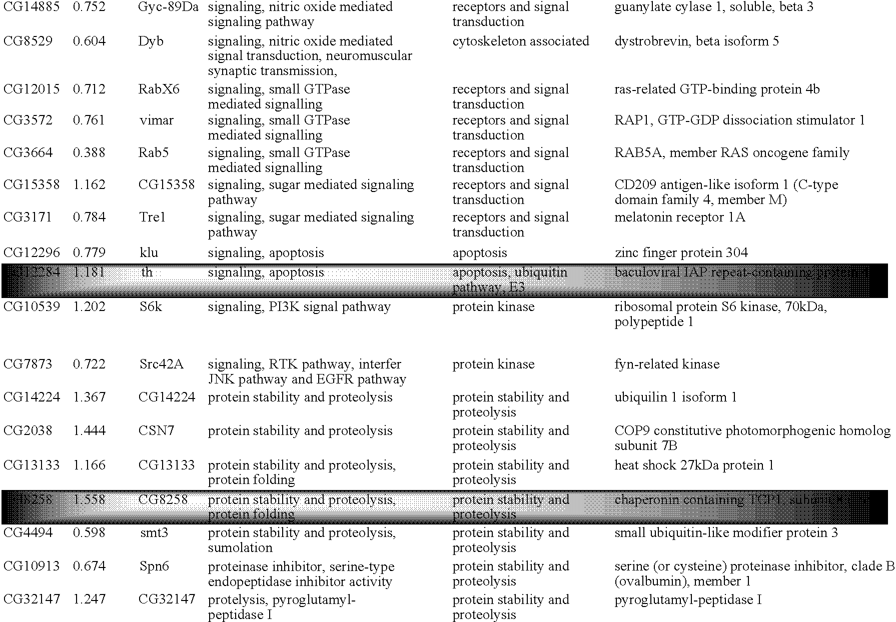

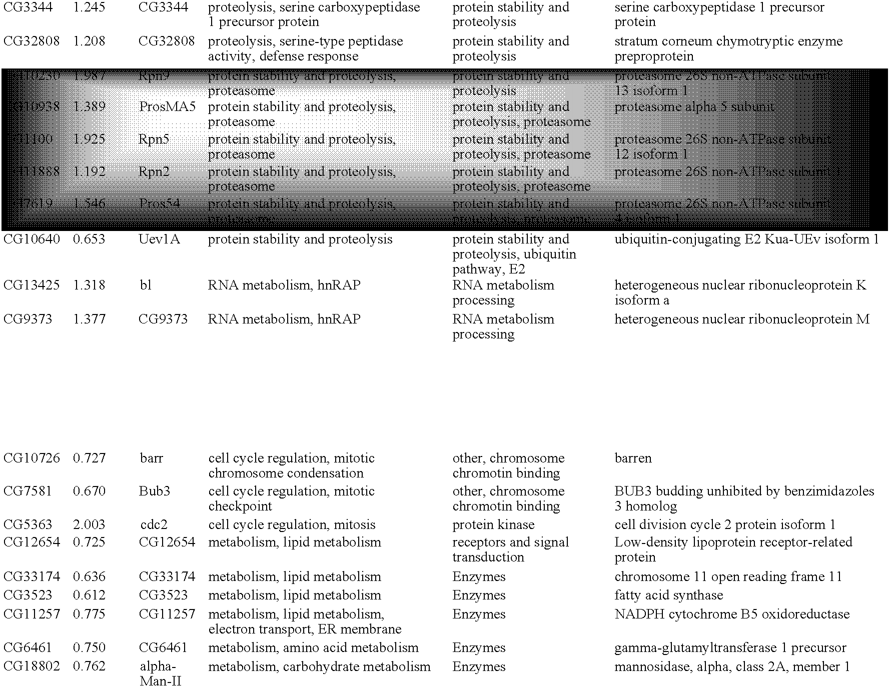

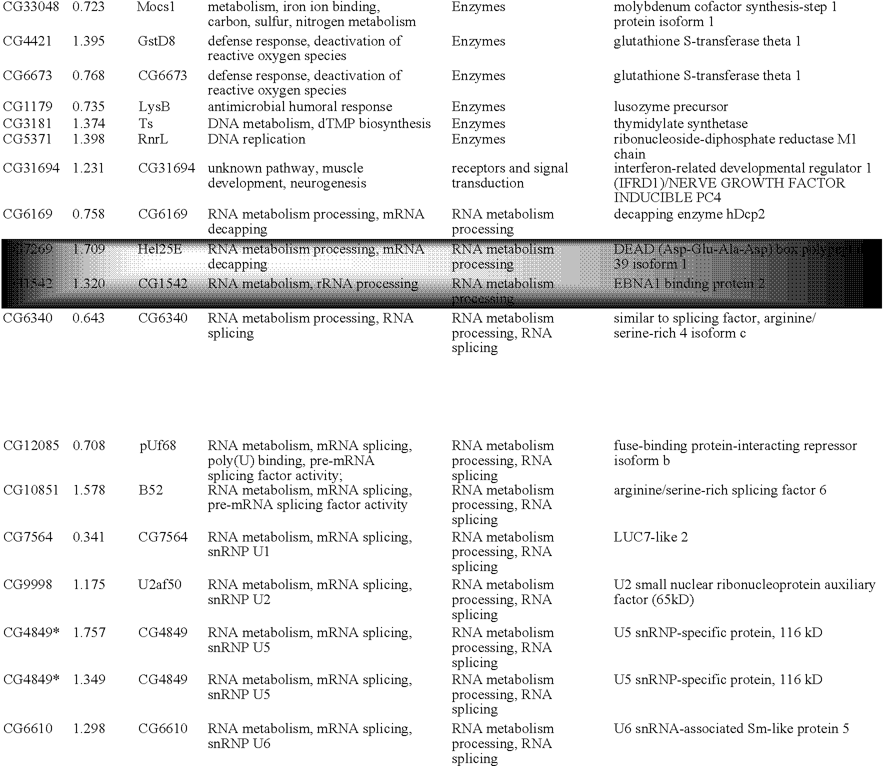

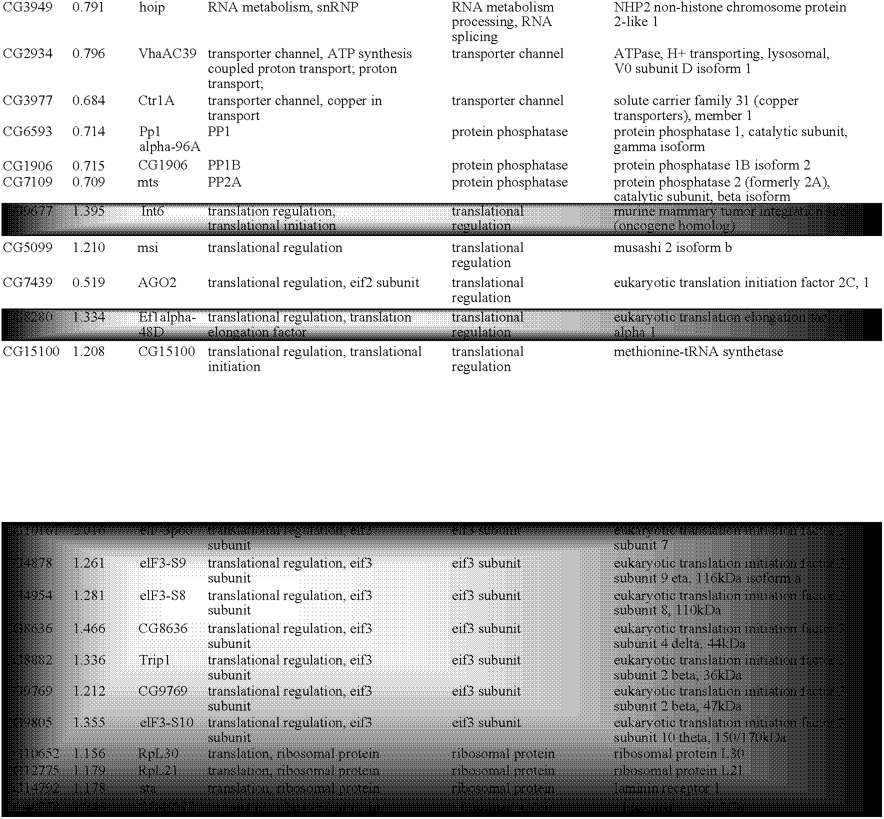

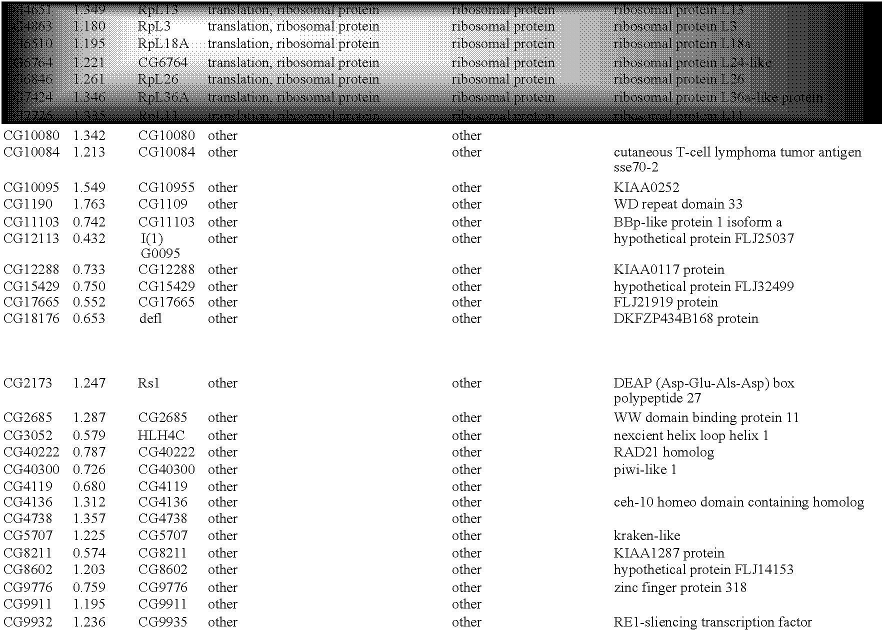

153 hits identified from whole genome RNAi screen are listed. For each dsRNA used, correspondent CG, CS fold change, gene name, biological process andl pathway, molecular function, human protein name are shown. CS_fold refers to a fold change of CS activity against LacZ control. Hits highlighted in grey are those with low renilla activity.
*two different dsRNA correspond to CG4849.

A “therapeutically effective amount” is the amount of drug sufficient to treat, prevent or ameliorate conditions associated with mitochondrial dysfunction, e.g.,.

A “transgenic” organism as used herein refers to an organism that has had extra genetic material inserted into its genome. As used herein, a “transgenic fly” includes embryonic, larval and adult forms of Drosophila that contain a DNA sequence from the same or another organism randomly inserted into their genome. Although Drosophila melanogaster is preferred, it is contemplated that any fly of the genus Drosophila may be used in the present invention.

The term “conditions associated with mitochondrial dysfunction,” as used herein includes but is not limited to neurodegenerative diseases (e.g., Parkinson's Disease, Alzheimer's Disease, Huntington's Disease), cardiovascular diseases, diabetes, age-related disorders, and cancers.

As used herein, “neurodegenerative diseases” include but are not limited to Huntington's disease, Parkinson's Disease, Alzheimer's Disease, dystonia, dementia, multiple sclerosis, Amyotrophic Lateral Sclerosis (ALS), and Creutzfeld-Jacob Disease.

As used herein, “cardiovascular diseases” include but are not limited to ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), and myocardial disease (e.g., myocarditis, congestive cardiomyopathy, and hypertrophic cariomyopathy).

As used herein, “age-related disorders” include conditions associated with aging, particularly aging processes in which mitochondria is implicated, and characterized by at least one of (i) increased reactive oxygen species (ROS) production, (ii) mitochondrial DNA (mtDNA) damage accumulation, and (iii) progressive respiratory chain dysfunction. “Age-related disorders” include but are not limited to incontinence, diabetes mellitus, bone and joint problems (e.g., osteoporosis), strokes, dementia, functional disability, cardiac and respiratory disorders, and neurodegenerative disorders.

As used herein, “cancers” include but are not limited to As used herein, the term “cancer” includes solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin, central nervous system including brain; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc. Both human and veterinary uses are contemplated.

As used herein, “ectopic” expression of the transgene refers to expression of the transgene in a tissue or cell or at a specific developmental stage where it is not normally expressed.

As used herein, “phenotype” refers to the observable physical or biochemical characteristics of an organism as determined by both genetic makeup and environmental influences.

As used herein, a “control fly” refers to fly that is of the same genotype as flies used in the methods of the present invention except that the control fly does not carry the mutation being tested for modification of phenotype.

As used herein, “elevated transcription of mRNA” refers to a greater amount of mRNA transcribed from the natural endogenous gene encoding a protein, e.g., a human protein set forth in TABLE I, compared to control levels. Elevated mRNA levels of a protein, e.g., a human protein disclosed on TABLE I, may be present in a tissue or cell of an individual suffering from a condition associated with mitochondrial dysfunction compared to levels in a subject not suffering from said condition. In particular, levels in a subject suffering from said condition may be at least about twice, preferably at least about five times, more preferably at least about 10 times, most preferably at least about 100 times the amount of mRNA found in corresponding tissues in humans who do not suffer from said condition. Such elevated level of mRNA may eventually lead to increased levels of protein translated from such mRNA in an individual suffering from said condition as compared to levels in a healthy individual.

Methods of obtaining transgenic organisms, including transgenic Drosophila, are well-known to one skilled in the art. For example, a commonly used reference for P-element mediated transformation is Spradling, Drosophila: A practical approach, Roberts, Ed., 175-197, IRL Press, Oxford, UK (1986). The EP element technology refers to a binary system, utilizing the yeast Gal4 transcriptional activator, that is used to ectopically regulate the transcription of endogenous Drosophila genes. This technology is described in Brand and Perrimon, Development, Vol. 118, No. 2, 401-415 (1993); and Rorth (1998), supra.

A “host cell”, as used herein, refers to a prokaryotic or eukaryotic cell that contains heterologous DNA that has been introduced into the cell by any means, e.g., electroporation, calcium phosphate precipitation, microinjection, transformation, viral infection and the like.

“Heterologous,” as used herein, means “of different natural origin” or represents a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g., a different copy number, or under the control of different regulatory elements.

A “vector” molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes”.

“Plasmids” generally are designated herein by a lower case p preceded and/or followed by capital letters and/or numbers, in accordance with standard naming conventions that are familiar to those of skill in the art. Starting plasmids disclosed herein are either commercially-available, publicly-available on an unrestricted basis, or can be constructed from available plasmids by routine application of well-known, published procedures. Many plasmids and other cloning and expression vectors that can be used in accordance with the present invention are well-known and readily-available to those of skill in the art. Moreover, those of skill, readily may construct any number of other plasmids suitable for use in the invention. The properties, construction and use of such plasmids, as well as other vectors, in the present invention will be readily apparent to those of skill from the present disclosure.

The term “isolated” means that the material is removed from its original environment, e.g., the natural environment, if it is naturally-occurring. For example, a naturally-occurring polynucleotide or polypeptide present in a living animal is not isolated, but the same polynucleotide or polypeptide, separated from some or all of the co-existing materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

As used herein, the term “transcriptional control sequence” or “expression control sequence” refers to DNA sequences, such as initiator sequences, enhancer sequences and promoter sequences, which induce, repress or otherwise control the transcription of a protein encoding nucleic acid sequences to which they are operably-linked. They may be tissue specific and developmental-stage specific.

A “human transcriptional control sequence” is a transcriptional control sequence normally found associated with the human gene encoding a polypeptide set forth in TABLE I of the present invention as it is found in the respective human chromosome.

A “non-human transcriptional control sequence” is any transcriptional control sequence not found in the human genome.

The term “polypeptide” is used, interchangeably herein, with the terms “polypeptides” and “protein(s)”.

A chemical derivative of a protein set forth in TABLE I of the invention is a polypeptide that contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, e.g., in Remington's Pharmaceutical Sciences, 16^{th} Edition, Mack Publishing Co., Easton, Pa. (1980).

The ability of a substance to “modulate” a protein set forth in TABLE I or a variant thereof, i.e., “a modulator of a protein selected from the group consisting of the proteins disclosed in TABLE I” includes, but is not limited to, the ability of a substance to inhibit or enhance the activity of said protein and/or variant thereof and/or inhibit or enhance the RNA expression of gene encoding said protein or variant. Such modulation could also involve affecting the ability of other proteins to interact with said protein, e.g., related regulatory proteins or proteins that are modified by said protein.

The term “agonist”, as used herein, refers to a molecule, i.e., modulator, which, directly or indirectly, may modulate a polypeptide, e.g., a polypeptide set forth in TABLE I or a variant thereof, and which increases the biological activity of said polypeptide. Agonists may include proteins, nucleic acids, carbohydrates or other molecules. A modulator that enhances gene transcription or a biological activity of a protein is something that increases transcription or stimulates the biochemical properties or activity of said protein, respectively.

The terms “antagonist” or “inhibitor” as used herein, refer to a molecule, i.e., modulator, which directly or indirectly may modulate a polypeptide or variant thereof, e.g., a polypeptide set forth in TABLE I, which blocks or inhibits the biological activity of said polypeptide. Antagonists and inhibitors may include proteins, nucleic acids, carbohydrates or other molecules. A modulator that inhibits gene expression or a biological activity of a protein is something that reduces gene expression or biological activity of said protein, respectively.

As generally referred to herein, a “protein or gene selected from the group consisting of the proteins disclosed in TABLE I” refers to the human form of the protein or gene. It is recognized, that polypeptides (or nucleic acids which encode those polypeptides) containing less than the described levels of sequence identity to proteins in TABLE I and arising as splice or allelic variants or that are modified by minor deletions, by conservative amino acid substitutions, by substitution of degenerate codons or the like, also are encompassed within the scope of the present invention. A variety of known algorithms are known in the art and have been disclosed publicly, and a variety of commercially-available software for conducting homology-based similarity searches are available and can be used to identify variants of proteins disclosed herein. Examples of such software includes, but are not limited to, FASTA (GCG Wisconsin Package), Bic_SW (Compugen Bioccelerator), BLASTN2, BLASTP2, BLASTD2 (NCBI) and Motifs (GCG). The BLAST algorithm is described in Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402. Suitable software programs are described, e.g., in Guide to Human Genome Computing, 2^{nd} edition, Bishop, Ed., Academic Press, San Diego, Calif. (1998); and The Internet and the New Biology: Tools for Genomic and Molecular Research, American Society for Microbiology, Peruski, Jr. and Harwood Peruski, Eds., Washington, D.C. (1997).

As described in detail herein, a genome-wide RNA interference screen was carried out in Drosophila S2 cells, to identify genes capable of modulating mitochondrial function. The mitochondrial enzyme citrate synthase (CS) activity, indicative of mitochondrial oxidative capacity, was used as the primary readout for the screen. The experiments represent the first attempt to discover regulators of mitochondrial biogenesis and functions at genomic scale using whole genome RNAi screen in higher eukaryotes. Furthermore, they represent the first time CS activity is used as a biochemical and functional readout, to systematically search for regulators of mitochondrial function.

CS is widely used as a common marker for mitochondrial oxidative activity. Knowledge of the regulatory mechanism of mitochondrial biogenesis extant in the field is mainly focused on PGC1-NRF pathway; however, the proximal promoter of human CS lacks binding sites for these transcription factors, suggesting a distinct pathway for CS gene regulation (Kraft C S, et al. (2006) American Journal of Phys.—Cell Phys. 290(4):C1119). Therefore, genome wide screen for genes that affect CS activity is expected to discover novel modulators and pathways for mitochondrial biogenesis and function (i.e., independent of the PGC1-NRF pathway in at least some instances).

The unbiased whole-genome RNAi screens were performed in Drosophila S2 cells. The Drosophila system was chosen for its high efficiency of RNAi, lower genetic redundancy, and large collection of mutations for in vivo analysis. In addition, Drosophila has been successfully utilized as a good model system for various mitochondrial diseases (Sanchez-Martinez A. et al. (2006) Biochim Biophys Acta. 1757: 1190).

The genome-wide RNAi screen identified high number of hits in mitochondrial related function, transcriptional regulation and signaling pathways, as discussed in detail herein. To further investigate their biological significance, a number of the hits were analyzed in vivo by using transgenic flies and fly mutants. The present studies reveal a novel function for HDAC6, and provide in vivo support for HDAC1, in modulating mitochondrial functions. Using fly mutations, in vivo evidence of modulation of mitochondrial function is provided for several known and novel genes, including AKAP, vimar, Src42A, klumpfuss, barren, and smt3. These hits are implicated in PKA signaling pathway, small GTPase mediated signaling pathway, RTK signaling pathway, apoptosis, mitotic regulation and simulation process.

The screen is highly specific and functionally relevant, as revealed by identification of multiple subunits for several complexes, enriched mitochondria-associated proteins, and several known regulators of mitochondrial functions.

### Mitochondrial Biogenesis

Mitochondrial biogenesis is a complex process that integrates developmental, metabolic, nutrient and environmental stimuli. Mitochondria proliferate as needed during myogenesis (Duguez S, et al. (2002) American Journal of Phys.—Endocr. & Metab. 282 (4):E802), upon growth hormone stimulation (Goglia F, et al. (1999) FEB S Letters 452(3):115), as a response to low temperature or exercise (Reznick R M, et al. (2006) Journal of Physiol. 574 (Pt 1):33). Mitochondria undergo constant and dynamic changes through fission, fusion and translocation (Chen H, et al. (2005) Human Mol. Gen. 14 Spec No. 2:R283) (Okamoto K, et al. (2005) Ann. Review of Gen. 39:503).

PGC1α (Peroxisome proliferator-activated receptor γ coactivator 1) is the first major regulator identified for mitochondrial biogenesis (Puigserver P, et al. (1998) Cell 92(6):829) (Wu Z, et al. (1999) Cell 98(1):115). The best characterized pathway for mitochondrial biogenesis is PGC1α—NRFs (nuclear respiratory factors) pathway. Environmental signals, such as cold or exercise, induce the expression of transcription coactivators of PGC-1 family (PGC-1α, PGC-1β, and PRC), which activate specific transcription factors (NRF-1, NRF-2, and ERRα) to induce the expression of respiratory genes, and the regulators of mtDNA transcription and replication. (Wu 1999) (Lehman J J, et al. (2000) Journal of Clin. Invest. 106(7):847) (Lin J, et al. (2002) JBC 277(3):1645) (Scarpulla R C (2002a) Biochimica et Biophysica Acta 1576(1-2):1) (Scarpulla R C (2002b) Gene 286(1):81) (Puigserver 1998) (Meirhaeghe A, et al. (2003) Biochem. Journal 373(Pt 1): 155) (Mootha V K, et al. (2004) PNAS 101(17):6570) (Schreiber S N, et al. (2004) PNAS 101 (17):6472) (Reznick 2006)

Other major regulators identified for mitochondrial biogenesis are CaMKIV (calcium/calmodulin-dependent protein kinase IV), AMPK (AMP-activated protein kinase) and NO (nitric oxide), which appear to regulate mitochondrial biogenesis through PGC1α-NRF pathway (Reznick 2006). CaMKIV was shown to be a positive regulator for mitochondrial biogenesis in myocytes (Wu H, et al. (2002) Science 296(5566):349). AMPK is a major regulator of mitochondrial biogenesis in response to chronic energy depletion (Zong H, et al. (2002) PNAS 99(25):15983) (Hardie D G (2004) Medicine & Science in Sports & Exercise 36(1):28) (Kahn B B, et al. (2005) Cell Metab. 1(1):15) (Hardie D G, et al. (2006) Physiology 21:48). NO produced by eNOS (endothelial nitric oxide synthase) has been shown to regulate mitochondrial biogenesis through PGC1α (Nisoli E, et al. (2003) Science 299(5608):89) (Nisoli E, et al. (2004b) PNAS 101 (47):16507). NO is an endogenous signaling molecule that activates guanylate cyclase to generate second messenger cGMP (cyclic GMP). (Moncada S, et al. (1991) Pharmaco. Reviews 43(2):109) (Alderton W K, et al. (2001) Biochem. Journal 357(Pt 3):593)

Additionally, PGC1α also binds nuclear hormone receptors, including retinoic acid receptor (RxR), the thyroid receptor (TR), the Peroxisome proliferator-activated receptor (PPAR). (Knutti D, et al. (2001) Trends in Endocrin. & Metab. 12(8):360) (Puigserver P, et al. (2003) Endocrine Reviews 24(1):78) Thus, PGC-1α may have additional functions through coactivating different receptors. It is noteworthy that a PGC1α-independent pathway for mitochondrial biogenesis may exist, as indicated by the fact that mitochondrial abundance and morphology appear normal in brown fat and liver from PGC1α knock-out mice, despite the hepatocytes from these mice having defects in hormone-induced gluconeogenesis and lower (17% reduced) O₂ consumption rate. (Lin J, et al. (2004) Cell 119(1):121)

Nucleic acid molecules of the human homologs of the target polypeptides disclosed herein may act as target gene antisense molecules, useful, e.g., in target gene regulation and/or as antisense primers in amplification reactions of target gene nucleic acid sequences. Further, such sequences may be used as part of ribozyme and/or triple-helix sequences or as targets for siRNA or double- or single-stranded RNA, which may be employed for gene regulation. Still further, such molecules may be used as components of diagnostic kits as disclosed herein.

In cases where an identified gene is the normal or wild type gene, this gene may be used to isolate mutant alleles of the gene. Such isolation is preferable in processes and disorders which are known or suspected to have a genetic basis. Mutant alleles may be isolated from individuals either known or suspected to have a genotype which contributes to conditions associated with mitochondrial dysfunction. Mutant alleles and mutant allele products may then be utilized in the diagnostic assay systems described herein.

A cDNA of the mutant gene may be isolated, e.g., by using PCR, a technique which is well-known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the complementary (cDNA) is then synthesized using an oligonucleotide that hybridizes specifically to the 5′ end of the normal gene. Using these two primers, the product is then amplified via PCR, cloned into a suitable vector, and subjected to DNA sequence analysis through methods well-known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probe to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of or known to carry the mutant allele. In this manner, gene products made by the putatively mutant tissue may be expressed and screened using standard antibody screening techniques in conjunction with antibodies raised against the normal gene product, as described below. For screening techniques, see, e.g., Antibodies: A Laboratory Manual, Harlow and Lane, Eds., Cold Spring Harbor Press, Cold Spring Harbor, N.Y. (1988). In cases where the mutation results in an expressed gene product with altered function, e.g., as a result of a mis-sense mutation, a polyclonal set of antibodies are likely to cross-react with the mutant gene product. Library clones detected via their reaction with such labeled antibodies can be purified and subjected to sequence analysis as described above.

The pharmaceutical compositions of the present invention may also comprise substances that inhibit the expression of a protein disclosed in TABLE I or variants thereof at the nucleic acid level. Such molecules include ribozymes, antisense oligonucleotides, triple-helix DNA, RNA aptamers, siRNA and/or double- or single-stranded RNA directed to an appropriate nucleotide sequence of nucleic acid encoding such a protein. These inhibitory molecules may be created using conventional techniques by one of skill in the art without undue burden or experimentation. For example, modifications, e.g., inhibition, of gene expression can be obtained by designing antisense molecules, DNA or RNA, to the control regions of the genes encoding the polypeptides discussed herein, i.e., to promoters, enhancers and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of gene expression may be achieved using “triple-helix” base-pairing methodology. Triple helix pairing is useful because it causes inhibition of the ability of the double-helix to open sufficiently for the binding of polymerases, transcription factors or regulatory molecules. Recent therapeutic advances using triplex-DNA have been described in the literature. See Gee et al., Molecular and Immunologic Approaches, Huber and Carr, Eds., Futura Publishing Co., Mt. Kisco, N.Y. (1994). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences. Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules. See Grassi and Marini, Ann Med, Vol. 28, No. 6, 499-510 (1996); and Gibson, Cancer Metastasis Rev, Vol. 15, No. 3, 287-299 (1996). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell. See Cotten and Birnstiel, EMBO J, Vol. 8, No. 12, 3861-3866 (1989). In particular, a ribozyme coding DNA sequence, designed according to conventional, well-known rules and synthesized, e.g., by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter, e.g., a glucocorticoid or a tetracycline response element, is also introduced into this construct so that ribozyme expression can be selectively controlled. For saturating use, a highly and constituently active promoter can be used. tDNA genes, i.e., genes encoding tRNAs, are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly, the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA [see Good et al., Gene Ther, Vol. 4, No. 1, 45-54 (1997)] that can specifically inhibit their translation.

Gene specific inhibition of gene expression may also be achieved using conventional double- or single-stranded RNA technologies. A description of such technology may be found in WO 99/32619, which is hereby incorporated by reference in its entirety. In addition, siRNA technology has also proven useful as a means to inhibit gene expression. See Cullen, Nat Immunol, Vol. 3, No. 7, 597-599 (2002); and Martinez et al., Cell, Vol. 110, No. 5, 563-574 (2002).

Antisense molecules, triple-helix DNA, RNA aptamers, dsRNA, ssRNA, siRNA and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters, such as T7 or SP6. Alternatively, cDNA constructs that synthesize antisense RNA constitutively or inducibly can be introduced into cell lines, cells or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well-known in the art.

Detection of mRNA levels of proteins disclosed herein may comprise contacting a biological sample or even contacting an isolated RNA or DNA molecule derived from a biological sample with an isolated nucleotide sequence of at least about 20 nucleotides in length that hybridizes under high-stringency conditions, e.g., 0.1×SSPE or SSC, 0.1% SDS, 65° C.) with the isolated nucleotide sequence encoding a polypeptide set forth in TABLE I. Hybridization conditions may be highly-stringent or less highly-stringent. In instances wherein the nucleic acid molecules are deoxyoligonucleotides (oligos), highly-stringent conditions may refer, e.g., to washing in 6×SSC/0.05% sodium pyrophosphate at 37° C. (for 14-base oligos), 48° C. (for 17-base oligos), 55° C. (for 20-base oligos) and 60° C. (for 23-base oligos). Suitable ranges of such stringency conditions for nucleic acids of varying compositions are described in Krause and Aaronson, Methods Enzymol, Vol. 200, 546-556 (1991) in addition to Maniatis et al., cited above.

In some cases, detection of a mutated form of the gene which is associated with a dysfunction will provide a diagnostic tool that can add to or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acids, in particular mRNA, for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences encoding a polypeptide disclosed in TABLE I or variants thereof. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers, Larin and Maniatis, Science, Vol. 230, No. 4731, 1242-1246 (1985). Sequence changes at specific locations may also be revealed by nuclease protection assays, such as RNase and S1 protection or the chemical cleavage method. See Cotton et al., Proc Natl Acad Sci USA, Vol. 85, 4397-4401 (1985). In addition, an array of oligonucleotides probes comprising nucleotide sequence encoding the polypeptides given by TABLE I, or variants or fragments of such nucleotide sequences can be constructed to conduct efficient screening of, e.g., genetic mutations. Array technology methods are well-known and have general applicability and can be used to address a variety of questions in molecular genetics including gene expression, genetic linkage and genetic variability. See, e.g., Chee et al., Science, Vol. 274, No. 5287, 610-613 (1996).

The diagnostic assays offer a process for diagnosing or determining a susceptibility to disease through detection of mutation in the gene of a polypeptide set forth in TABLE I by the methods described. In addition, such diseases may be diagnosed by methods comprising determining from a sample derived from a subject an abnormally decreased or increased level of polypeptide or mRNA. Decreased or increased expression can be measured at the RNA level using any of the methods well-known in the art for the quantitation of polynucleotides, such as, e.g., nucleic acid amplification, for instance, PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

The present invention also discloses a diagnostic kit for detecting mRNA levels (or protein levels) which comprises:

(a) a polynucleotide of a polypeptide set forth in TABLE I or a fragment thereof;

(b) a nucleotide sequence complementary to that of paragraph (a);

(c) a polypeptide of TABLE I of the present invention encoded by the polynucleotide of paragraph (a);

(d) an antibody to the polypeptide of paragraph (c); and

(e) an RNAi sequence complementary to that of paragraph (a).

It will be appreciated that in any such kit, any of the substances in (a), (b), (c), (d) or (e) may comprise a substantial component. Such a kit will be of use in diagnosing a disease or susceptibility to a disease, particularly to a condition associated with mitochondrial dysfunction.

The differences in the cDNA or genomic sequence between affected and unaffected individuals can also be determined. If a mutation is observed in some or all of the affected individuals but not in any normal individuals, then the mutation is likely to be the causative agent of the disease.

Gene Therapy

In another aspect, nucleic acids comprising a sequence encoding a polypeptide set forth in TABLE I or a functional-derivative thereof, may be administered to promote normal biological activity, e.g., normal mitochondrial biogenesis, by way of gene therapy. Gene therapy refers to therapy performed by the administration of a nucleic acid to a subject. In this aspect of the invention, the nucleic acid produces its encoded protein that mediates a therapeutic effect by, e.g., promoting normal mitochondrial biogenesis.

Any of the methods for gene therapy available in the art can be used according to the present invention. Exemplary methods are described below.

In a preferred aspect, the therapeutic comprises a nucleic acid encoding any polypeptide given by TABLE I. Commonly the nucleic acid is part of an expression vector that expresses a protein given by TABLE I, a fragment or chimeric protein thereof and variants thereof in a suitable host. In particular, such a nucleic acid has a promoter operably-linked to a coding region encoding a protein of TABLE I, said promoter being inducible or constitutive, and, optionally, tissue-specific. In another particular aspect, a nucleic acid molecule is used in which the protein coding sequences for any of TABLE I and any other desired sequences are flanked by regions that promote homologous recombination at a desired site in the genome, thus providing for intrachromosomal expression of the nucleic acid encoding the particular protein. See Koller and Smithies, Proc Natl Acad Sci USA, Vol. 86, No. 22, 8932-8935 (1989); and Zijlstra et al., Nature, Vol. 342, No. 6248, 435-438 (1989).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific aspect, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment, e.g., a gene gun; Biolistic, Dupont, or coating with lipids or cell-surface receptors or transfecting agents, encapsulation in liposomes, microparticles or microcapsules, or by administering it in linkage to a peptide which is known to enter the nucleus, by administering it in linkage to a ligand subject to receptor-mediated endocytosis (see, e.g., U.S. Pat. Nos. 5,166,320; 5,728,399; 5,874,297 and 6,030,954, all of which are incorporated by reference herein in their entirety), which can be used to target cell types specifically expressing the receptors, etc. In another aspect, a nucleic acid-ligand complex can be formed in which the ligand comprises a fusogenic viral peptide to disrupt endosomes, allowing the nucleic acid to avoid lysosomal degradation. In yet another aspect, the nucleic acid can be targeted in vivo for cell specific uptake and expression, by targeting a specific receptor. See, e.g., PCT Publications WO 92/06180; WO 92/22635; WO 92/20316; WO 93/14188 and WO 93/20221. Alternatively, the nucleic acid can be introduced intracellularly and incorporated within host cell DNA for expression, by homologous recombination. See, e.g., U.S. Pat. Nos. 5,413,923; 5,416,260 and 5,574,205; and Zijlstra et al. (1989), supra.

In a specific aspect, a viral vector that contains a nucleic acid encoding a polypeptide of TABLE I is used. For example, a retroviral vector can be used. See, e.g., U.S. Pat. Nos. 5,219,740; 5,604,090 and 5,834,182. These retroviral vectors have been modified to delete retroviral sequences that are not necessary for packaging of the viral genome and integration into host cell DNA. The nucleic acid for the polypeptide of TABLE I to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557 and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496 and 5,948,675, all of which are incorporated by reference herein in their entirety.

Another approach to gene therapy involves transferring a gene to cells in tissue culture by such methods as electroporation, lipofection, calcium phosphate mediated transfection or viral infection. Usually, the method of transfer includes the transfer of a selectable marker to the cells. The cells are then placed under selection to isolate those cells that have taken up and are expressing the transferred gene. Those cells are then delivered to a patient.

The resulting recombinant cells can be delivered to a patient by various methods known in the art. In a preferred aspect, epithelial cells are injected, e.g., subcutaneously. In another aspect, recombinant skin cells may be applied as a skin graft onto the patient. Recombinant blood cells, e.g., hematopoietic stem or progenitor cells, are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type and include, but are not limited to, epithelial cells, endothelial cells, keratinocytes, fibroblasts, muscle cells, hepatocytes; blood cells, such as T lymphocytes, B lymphocytes, monocytes, macrophages, neutrophils, eosinophils, megakaryocytes, granulocytes; various stem or progenitor cells, in particular, hematopoietic stem or progenitor cells, e.g., as obtained from bone marrow, umbilical cord blood, peripheral blood, fetal liver, etc.

In a preferred aspect, the cell used for gene therapy is autologous to the patient.

In an aspect, in which recombinant cells are used in gene therapy, the nucleic acid of a polypeptide set forth in TABLE I is introduced into the cells such that it is expressible by the cells or their progeny, and the recombinant cells are then administered in vivo for therapeutic effect. In a specific aspect, stem or progenitor cells are used. Any stem cells and/or progenitor cells that can be isolated and maintained in vitro can potentially be used in accordance with this aspect of the present invention. Such stem cells include, but are not limited to, hematopoietic stem cells (HSC), stem cells of epithelial tissues such as the skin and the lining of the gut, embryonic heart muscle cells, liver stem cells (see, e.g., WO 94/08598) and neural stem cells. See Stemple and Anderson, Cell, Vol. 71, No. 6, 973-985 (1992).

Epithelial stem cells (ESCs) or keratinocytes can be obtained from tissues, such as the skin and the lining of the gut by known procedures. See Rheinwald, Methods Cell Biol, Vol. 21A, 229-254 (1980). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture. See Pittelkow and Scott, Mayo Clin Proc, Vol. 61, No. 10, 771-777 (1986). If the ESCs are provided by a donor, a method for suppression of host versus graft reactivity, e.g., irradiation, drug or antibody administration to promote moderate immunosuppression, can also be used.

With respect to HSCs, any technique which provides for the isolation, propagation and maintenance in vitro of HSCs can be used in this aspect of the invention. Techniques by which this may be accomplished include:

(a) the isolation and establishment of HSC cultures from bone marrow cells isolated from the future host or a donor; or

(b) the use of previously established long-term HSC cultures, which may be allogeneic or xenogeneic.

Non-autologous HSC are used preferably in conjunction with a method of suppressing transplantation immune reactions of the future host/patient. In a particular aspect of the present invention, human bone marrow cells can be obtained from the posterior iliac crest by needle aspiration. See, e.g., Kodo, Gale and Saxon, J Clin Invest, Vol. 73, No. 5, 1377-1384 (1984). In a preferred aspect of the present invention, the HSCs can be made highly enriched or in substantially pure form. This enrichment can be accomplished before, during or after long-term culturing, and can be done by any techniques known in the art. Long-term cultures of bone marrow cells can be established and maintained by using, e.g., modified Dexter cell culture techniques [see Dexter et al., J Cell Physiol, Vol. 91, No. 3, 335-344 (1977)] or Witlock-Witte culture techniques. See Witlock and Witte, Proc Natl Acad Sci USA, Vol. 79, No. 11, 3608-3612 (1982).

In a specific aspect, the nucleic acid to be introduced for purposes of gene therapy comprises an inducible promoter operably-linked to the coding region, such that expression of the nucleic acid is controllable by controlling the presence or absence of the appropriate inducer of transcription.

Pharmaceutical Compositions

An additional aspect of the invention relates to the administration of a pharmaceutical composition, in conjunction with a pharmaceutically acceptable carrier, excipient or diluent, for any of the therapeutic effects discussed above. Such pharmaceutical compositions may comprise, for example, a polypeptide set forth in TABLE I, antibodies to that polypeptide, mimetics, agonists, antagonists, inhibitors or other modulators of function of a polypeptide given by TABLE I or a gene therefore. The compositions may be administered alone or in combination with at least one other agent, such as stabilizing compound, which may be administered in any sterile, biocompatible pharmaceutical carrier including, but not limited to, saline, buffered saline, dextrose and water. The compositions may be administered to a patient alone, or in combination with other agents, drugs or hormones.

In addition, any of the therapeutic proteins, antagonists, antibodies, agonists, antisense sequences or other modulators described above may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment, prevention or amelioration of pathological conditions associated with abnormalities in mitochondrial biogenesis. Using this approach, one may be able to achieve therapeutic efficacy with lower dosages of each agent, thus reducing the potential for adverse side effects. Antagonists, agonists and other modulators of the human polypeptides set forth in TABLE I and genes encoding said polypeptides and variants thereof may be made using methods which are generally known in the art.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well-known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato or other plants; cellulose, such as methyl cellulose, hydroxypropylmethyl-cellulose or sodium carboxymethylcellulose; gums including arabic and tragacanth; and proteins, such as gelatin and collagen. If desired, disintegrating or solubilizing agents may be added, such as the cross-linked polyvinyl pyrrolidone, agar, alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches; lubricants, such as talc or magnesium stearate; and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution or physiologically-buffered saline. Aqueous injection suspensions may contain substances that increase the viscosity of the suspension, such as sodium carboxymethyl cellulose, sorbitol or dextran. Additionally, suspensions of the active compounds may be prepared as appropriate oily injection suspensions. Suitable lipophilic solvents or vehicles include fatty oils, such as sesame oil; or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly-concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with many acids including, but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder that may contain any or all of the following: 1-50 mM histidine, 0.1-2% sucrose and 2-7% mannitol, at a pH range of 4.5-5.5, that is combined with buffer prior to use.

After pharmaceutical compositions have been prepared, they can be placed in an appropriate container and labeled for treatment of an indicated condition. Such labeling would include amount, frequency and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs or pigs. The animal model may also be used to determine the appropriate concentration range and route of administration. Such information can then be used to determine useful doses and routes for administration in humans.

A therapeutically-effective dose refers to that amount of active ingredient which ameliorates the symptoms or condition. Therapeutic efficacy and toxicity may be determined by standard pharmaceutical procedures in cell cultures or experimental animals, e.g., the dose therapeutically effective in 50% of the population (ED₅₀) and the dose lethal to 50% of the population (LD₅₀). The dose ratio between toxic and therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities and tolerance/response to therapy. Long-acting pharmaceutical compositions may be administered every 3-4 days, every week or once every two weeks depending on half-life and clearance rate of the particular formulation.

Normal dosage amounts may vary from 0.1-100,000 mg, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569 and 6,051,561.

Antibodies

A further aspect of the present invention relates to a method to treat, prevent or ameliorate conditions associated with mitochondrial dysfunction, comprising administering to a subject in need thereof an effective amount of a modulator of a protein selected from the group consisting of the proteins disclosed in TABLE I and/or variants thereof. In one aspect, the modulator comprises one or more antibodies to said protein, variant or fragments thereof, wherein said antibodies or fragments thereof can inhibit a biological activity of said protein or variant in said subject.

The proteins of TABLE I can be used as immunogens to generate antibodies using standard techniques for polyclonal and monoclonal antibody preparation. The full length polypeptide or protein can be used or, alternatively, the invention provides antigenic peptide fragments for use as immunogens. The antigenic peptide, or epitope, of a protein of TABLE I comprises at least 8 (preferably 10, 15, 20, or 30) amino acid residues of the amino acid sequence of any of the proteins of TABLE I, and encompasses an epitope of the protein such that an antibody raised against the peptide forms a specific immune complex with the protein.

Preferred epitopes encompassed by the antigenic peptide are regions that are located on the surface of the protein, e.g., hydrophilic regions. Hydropathy plots or similar analyses can be used to identify hydrophilic regions.

Described herein are methods for the production of antibodies capable of specifically recognizing one or more differentially expressed gene epitopes. Such antibodies may include, but are not limited to, polyclonal antibodies, monoclonal antibodies (mAbs), humanized or chimeric antibodies, single-chain antibodies, Fab fragments, F(ab′)₂ fragments, fragments produced by a Fab expression library, anti-idiotypic (anti-Id) antibodies and epitope-binding fragments of any of the above. Such antibodies may be used, e.g., in the detection of a target protein in a biological sample, or alternatively, as a method for the inhibition of a biological activity of the protein. Thus, such antibodies may be utilized as part of disease treatment methods, and/or may be used as part of diagnostic techniques whereby patients may be tested, e.g., for abnormal levels of polypeptides set forth in TABLE I, or for the presence of abnormal forms of these polypeptides.

For the production of antibodies to the polypeptides given by TABLE I or variants thereof, various host animals may be immunized by injection with these polypeptides, or a portion thereof. Such host animals may include but are not limited to rabbits, mice, goats, chickens and rats, to name but a few. Various adjuvants may be used to increase the immunological response, depending on the host species including, but not limited to, Freund's (complete and incomplete); mineral gels, such as aluminum hydroxide; surface active substances, such as lysolecithin, pluronic polyols, polyanions, peptides, oil emulsions, keyhole limpet hemocyanin and dinitrophenol; and potentially useful human adjuvants, such as bacille Calmette-Guerin (BCG) and Corynebacterium parvum.

As used herein, the term antibody refers to immunoglobulin molecules and immunologically active portions of immunoglobulin molecules, i.e., molecules that contain an antigen binding site which specifically binds an antigen, as well as fragments thereof. Antibodies can be fragmented using conventional techniques and the fragments screened for utility in the same manner as described herein below for whole antibodies. Examples of immunologically active portions of immunoglobulin molecules include F(ab) and F(ab′)2 fragments which can be generated by treating the antibody with an enzyme such as pepsin. The invention provides polyclonal and monoclonal antibodies.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with a polypeptide given by TABLE I, or a portion thereof, supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique [see Kohler and Milstein, Nature, Vol. 256, No. 5517, 495-497 (1975) and U.S. Pat. No. 4,376,110]; the human B-cell hybridoma technique [see Kosbor et al., Immunol Today, Vol. 4, 72 (1983) and Cole et al., Proc Natl Acad Sci USA, Vol. 80, 2026-2030 (1983)]; and the EBV-hybridoma technique. See Cole et al., Monoclonal Antibodies And Cancer Therapy, Alan R. Liss, Inc., 77-969 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well known techniques, such as protein A chromatography to obtain the IgG fraction. At an appropriate time after immunization, e.g., when the specific antibody titers are highest, antibody producing cells can be obtained from the subject and used to prepare monoclonal antibodies by standard techniques, such as the hybridoma technique originally described by Kohler and Milstein (1975) Nature 256:495 497, the human B-cell hybridoma technique (Kozbor et al. (1983) Immunol. Today 4:72), the EBV hybridoma technique (Cole et al. (1985), Monoclonal Antibodies and Cancer Therapy, Alan R. Liss, Inc., pp. 77 96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27 9400 01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370 1372; Hay et al. (1992) Hum. Antibod. Hybridomas 3:81 85; Huse et al. (1989) Science 246:1275 1281; Griffiths et al. (1993) EMBO J. 12:725 734.

Additionally, recombinant antibodies, such as chimeric and humanized monoclonal antibodies, comprising both human and non human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041 1043; Liu et al. (1987) Proc. Natl. Acad. Sci. USA 84:3439 3443; Liu et al. (1987) J. Immunol. 139:3521 3526; Sun et al. (1987) Proc. Natl. Acad. Sci. USA 84:214 218; Nishimura et al. (1987) Canc. Res. 47:999 1005; Wood et al. (1985) Nature 314:446 449; and Shaw et al. (1988) J. Natl. Cancer Inst. 80:1553 1559); Morrison (1985) Science 229:1202 1207; Oi et al. (1986) Bio/Techniques 4:214; U.S. Pat. No. 5,225,539; Jones et al. (1986) Nature 321:552 525; Verhoeyan et al. (1988) Science 239:1534; and Beidler et al. (1988) J. Immunol. 141:4053 4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899 903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Alternatively, techniques described for the production of single-chain antibodies [U.S. Pat. No. 4,946,778; Bird, Science, Vol. 242, 423-426 (1988); Huston et al., Proc Natl Acad Sci USA, Vol. 85, No. 16, 5879-5883 (1988); and Ward et al., Nature, Vol. 334, 544-546 (1989)] can be adapted to produce differentially-expressed gene, single-chain antibodies. Single-chain antibodies are formed by linking the heavy- and light-chain fragments of the Fv region via an amino acid bridge, resulting in a single-chain polypeptide.

Antibody fragments that recognize specific epitopes may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed [see Huse et al., Science, Vol. 246, No. 4935, 1275-1281 (1989)] to allow rapid and easy identification of monoclonal Fab fragments with the desired specificity.

As contemplated herein, an antibody of the present invention can be preferably used in a diagnostic kit for detecting levels of a protein disclosed in TABLE I or antigenic variants thereof in a biological sample, as well as in a method to diagnose subjects suffering from a condition associated with mitochondrial dysfunction who may be suitable candidates for treatment with modulators to a protein selected from the group consisting of the proteins disclosed in TABLE I. Preferably, said detecting step comprises contacting said appropriate tissue cell, e.g., biological sample, with an antibody which specifically binds to a polypeptide given by TABLE I, or fragments or variants thereof and detecting specific binding of said antibody with a polypeptide in said appropriate tissue, cell or sample wherein detection of specific binding to a polypeptide indicates the presence of a polypeptide set forth in TABLE I or a fragment thereof.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for a polypeptide given by TABLE I, or fragments or variants thereof.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, β-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of the polypeptide of TABLE I or variant which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually-detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Fusion Proteins

The invention provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in frame to each other. The heterologous polypeptide can be fused to the N terminus or C terminus of the polypeptide of the invention.

One useful fusion protein is a GST fusion protein in which the polypeptide of the invention is fused to the C terminus of GST sequences. Such fusion proteins can facilitate the purification of a recombinant polypeptide of the invention.

In another embodiment, the fusion protein contains a heterologous signal sequence at its N terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction may be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in frame to the polypeptide of the invention.

RNAi

The invention provides small interfering ribonucleic acid sequences (siRNA), as well as compositions and methods for inhibiting the expression of a gene or genes which encode the proteins of TABLE I in a cell or mammal using siRNA. The invention also provides compositions and methods for treating pathological conditions and diseases in a mammal caused by the aberrant expression of a gene or genes which encode proteins which modulate mitochondrial biogenesis (e.g., the proteins of TABLE I), or caused by the aberrant signaling of pathways of which said genes are integral members, using siRNA. siRNA directs the sequence-specific degradation of mRNA through a process known as RNA interference (RNAi).

The siRNA of the invention comprises an RNA strand (the antisense strand) having a region which is less than 30 nucleotides in length, generally 19-24 nucleotides in length, and is substantially complementary to at least part of an mRNA transcript of the gene or genes which encode the proteins of TABLE I. The use of these siRNAs enables the targeted degradation of mRNAs of genes that are implicated in, e.g., mitochondrial biogenesis.

The siRNA molecules according to the present invention mediate RNA interference (“RNAi”). The term “RNAi” is well known in the art and is commonly understood to mean the inhibition of one or more target genes in a cell by siRNA with a region which is complementary to the target gene. Various assays are known in the art to test siRNA for its ability to mediate RNAi (see for instance Elbashir et al., Methods 26 (2002), 199-213). The effect of the siRNA according to the present invention on gene expression will typically result in expression of the target gene being inhibited by at least 10%, 33%, 50%, 90%, 95% or 99% when compared to a cell not treated with the RNA molecules according to the present invention.

“siRNA” or “small-interfering ribonucleic acid” according to the invention has the meanings known in the art, including the following aspects. The siRNA consists of two strands of ribonucleotides which hybridize along a complementary region under physiological conditions. The strands are separate but they may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

The siRNA molecules in accordance with the present invention comprise a double-stranded region which is substantially identical to a region of the mRNA of the target gene. A region with 100% identity to the corresponding sequence of the target gene is suitable. This state is referred to as “fully complementary.” However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Another factor affecting the efficiency of the RNAi reagent is the target region of the target gene. The region of a target gene effective for inhibition by the RNAi reagent may be determined by experimentation. A suitable mRNA target region would be the coding region. Also suitable are untranslated regions, such as the 5′-UTR, the 3′-UTR, and splice junctions. For instance, transfection assays as described in Elbashir S. M. et al, 2001 EMBO J., 20, 6877-6888 may be performed for this purpose. A number of other suitable assays and methods exist in the art which are well known to the skilled person.

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention confer a high in vivo stability suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918 and will not be repeated here. Suitable modifications for oral delivery are more specifically set out in the Examples and description herein. Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides with for instance phosphorothioates, chiral phosphorothioates or phosphorodithioates). Finally, end modifications sometimes referred to herein as 3′ caps or 5′ caps may be of significance. Caps may consist of more complex chemistries which are known to those skilled in the art.

In one embodiment, the invention provides double-stranded ribonucleic acid (dsRNA) molecules for inhibiting the expression of the gene or genes which encode the proteins of TABLE I. The dsRNA comprises at least two sequences that are complementary to each other. The dsRNA comprises a sense strand comprising a first sequence and an antisense strand comprising a second sequence. The antisense strand comprises a nucleotide sequence which is substantially complementary to at least part of an mRNA encoding the gene or genes which encode the proteins of TABLE I, and the region of complementarity is less than 30 nucleotides in length, generally 19-24 nucleotides in length. The dsRNA, upon contacting with a cell expressing the gene or genes which encode the proteins of TABLE I, inhibits the expression of said genes by at least 40%.

In another embodiment, the invention provides a cell comprising one of the dsRNAs of the invention. The cell is generally a mammalian cell, such as a human cell.

In another embodiment, the invention provides a pharmaceutical composition for inhibiting the expression of the gene or genes responsible for mitochondrial biogenesis in an organism (e.g., the genes which express the proteins of TABLE I), generally a human subject, comprising one or more of the dsRNA of the invention and a pharmaceutically acceptable carrier or delivery vehicle.

In another embodiment, the invention provides a method for inhibiting the expression of the gene or genes responsible for mitochondrial biogenesis (e.g., the genes which express the proteins of TABLE I) in a cell, comprising the following steps:

(a) introducing into the cell a double-stranded ribonucleic acid (dsRNA), wherein the dsRNA comprises at least two sequences that are complementary to each other. The dsRNA comprises a sense strand comprising a first sequence and an antisense strand comprising a second sequence. The antisense strand comprises a region of complementarity which is substantially complementary to at least a part of a mRNA encoding the gene or genes responsible for mitochondrial biogenesis (e.g., the genes which express the proteins of TABLE I), and wherein the region of complementarity is less than 30 nucleotides in length, generally 19-24 nucleotides in length, and wherein the dsRNA, upon contact with a cell expressing the gene or genes responsible for mitochondrial biogenesis (e.g., the genes which express the proteins of TABLE I), inhibits expression of said genes by at least 40%; and

(b) maintaining the cell produced in step (a) for a time sufficient to obtain degradation of the mRNA transcript of the gene or genes responsible for mitochondrial biogenesis (e.g., the genes which express the proteins of TABLE I), thereby inhibiting expression of said genes in the cell.

In another embodiment, the invention provides vectors for inhibiting the expression of the gene or genes responsible for mitochondrial biogenesis (e.g., the genes which express the proteins of TABLE I) in a cell, comprising a regulatory sequence operably linked to a nucleotide sequence that encodes at least one strand of one of the siRNA of the invention.

Inhibitory nucleic acid compounds of the present invention may be synthesized by conventional means on a commercially available automated DNA synthesizer, e.g. an Applied Biosystems (Foster City, Calif.) model 380B, 392 or 394 DNA/RNA synthesizer, or like instrument. Phosphoramidite chemistry may be employed. The inhibitory nucleic acid compounds of the present invention may also be modified, for instance, nuclease resistant backbones such as e.g., phosphorothioate, phosphorodithioate, phosphoramidate, or the like, described in many references may be used. The length of the inhibitory nucleic acid has to be sufficient to ensure that the biological activity is inhibited. Thus, for instance in case of antisense oligonucleotides, has to be sufficiently large to ensure that specific binding will take place only at the desired target polynucleotide and not at other fortuitous sites. The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, and the like. Preferably, the antisense oligonucleotides of the invention have lengths in the range of about 15 to 40 nucleotides. More preferably, the oligonucleotide moieties have lengths in the range of about 18 to 25 nucleotides.

Double-stranded RNA, i.e., sense-antisense RNA, also termed small interfering RNA (siRNA) molecules, can also be used to inhibit the expression of nucleic acids for the gene or genes responsible for mitochondrial biogenesis (e.g., the genes which express the proteins of TABLE I). RNA interference is a method in which exogenous, short RNA duplexes are administered where one strand corresponds to the coding region of the target mRNA (Elbashir et al. (2001) Nature 411: 494). Upon entry into cells, siRNA molecules cause not only degradation of the exogenous RNA duplexes, but also of single-stranded RNAs having identical sequences, including endogenous messenger RNAs. Accordingly, siRNA may be more potent and effective than traditional antisense RNA methodologies since the technique is believed to act through a catalytic mechanism. Preferred siRNA molecules are typically from 19 to 25 nucleotides long, preferably about 21 nucleotides in length. Effective strategies for delivering siRNA to target cells include, for example, transduction using physical or chemical transfection.

Alternatively siRNAs may be expressed in cells using, e.g., various PolIII promoter expression cassettes that allow transcription of functional siRNA or precursors thereof. See, for example, Scherr et al. (2003) Curr. Med. Chem. 10(3):245; Turki et al. (2002) Hum. Gene Ther. 13(18):2197; Cornell et al. (2003) Nat. Struct. Biol. 10(2):91. The invention also covers other small RNAs capable of mediating RNA interference (RNAi) such as for instance micro-RNA (miRNA) and short hairpin RNA (shRNA).

### EXAMPLES

### Example 1

### Whole Genome RNAi Screen

A cell based assay was developed for RNAi screening using mitochondrial citrate synthase (CS) activity as a readout. CS catalyzes the first reaction in the Krebs cycle (TCA cycle) to convert acetyl-CoA and oxaloacetate to citrate. CS activity is a common marker for mitochondrial oxidative activity. It has been shown that CS activity and mitochondrial DNA (mtDNA) content are positively correlated in muscle. (Wang, H., et al. (1999) European Journal of Applied Physiology & Occupational Physiology 80: 22) (Moyes, C. D., et al. (1997) American Journal of Physiology 272:C1345) As a mitochondrial functional marker, CS activity is a physiological relevant readout for mitochondrial function. Measuring CS activity kinetically ensures greater accuracy than an endpoint readout.

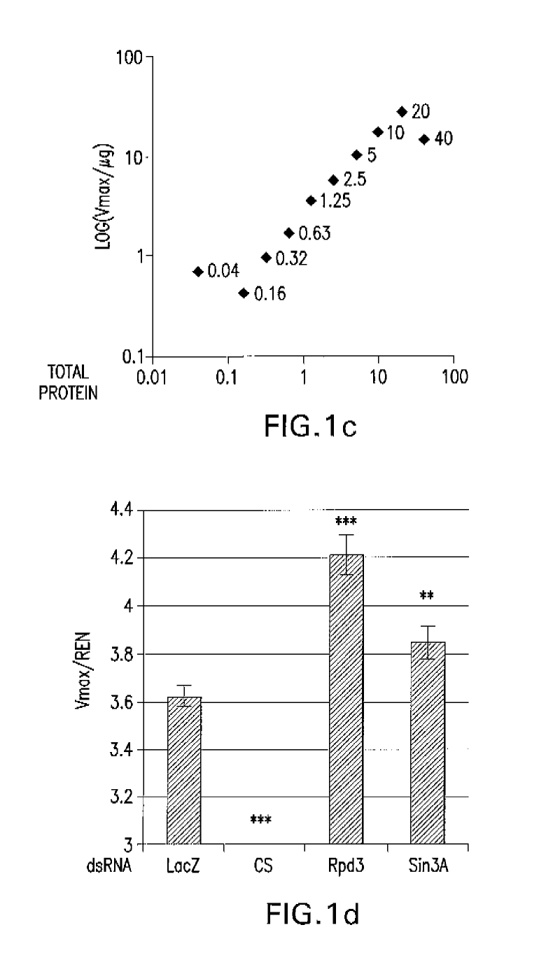

To determine whether CS activity could be adapted for high throughput RNAi screen, it was first tested whether CS could be modulated by RNAi in S2 cells. CS activities were highly reduced by dsRNA against CS gene in a dosage dependent manner (FIG. 1a). The stability and linearity of the CS assay were tested. CS enzyme activity was stable for at least 644 seconds (FIG. 1b) under the assay condition. This time window was sufficient for 15 readings of 384 samples (one microwell plate) and for calculating the Vmax. There was also a good linear range of CS activity when using 0.16 μg to 20 μg total protein of the lysates. The dynamic range of the CS assay was sufficient for a high throughput screen (FIG. 1c). Finally, to find positive controls for the screen and estimate the assay window, previously reported mitochondrial regulators were evaluated. Although CS is widely used as a common marker for mitochondrial function, little is known for its regulation (Kraft 2006). Sin3A, cofactor of HDAC1 (Rpd3), was reported to negatively regulate mitochondrial mass in Drosophila cells (Pile, L. A., et al. (2003) JBC 278: 37840). Rpd3 and Sin3A were then tested for their effects on CS activity. A 12% increase in CS activity by Rpd3 RNAi and 6% by Sin3A RNAi were detected (FIG. 1d). Taken together, CS activity has been tested and adapted for a high throughput screen.

The dsRNA collection used for these studies was obtained from Ambion, Inc. This collection has normalized dsRNA concentration in each well and represents 13,071 annotated genes from Drosophila genome, including all fly genes with human homologues. It was reformatted from the original 96 well plates to 384 well plates. The entire collection consists of thirty-six 384-well plates. RNAi were performed for 72 plates (36 plates in duplicate). Each well has 5 ul of dsRNA at concentration of 50 ng/ul. Before the screen, 5 ul of control dsRNAs were loaded in each empty well at 50 ng/ul in four replicates in each plate. The control CS dsRNA were synthesized from cDNA (HFA18328) collected by Heidelberg Fly Array (Hild, M., et al. (2003) Genome Biology 5: R3) using Megascript RNAi kit (Ambion, cat#1626). In order to normalize for RNAi mediated gene knockdown effects on cell growth and viability, a stable cell line bearing a renilla luciferase reporter construct was used in the experiments, and renilla luciferase activity was used to normalize citrate synthase activity. The renilla luciferase reporter gene is expressed by a basal promoter derived from hsp70 gene after removal of the CMV promoter in phRL-CMV plasmid.

For RNAi experiment, S2 cells were harvested, washed once in 1×PBS and re-suspended in serum free medium. 10,000 cells in 10 ul serum free medium were loaded in each well that contains dsRNA. After 1 hr incubation, 30 ul complete medium was added in each well, and then the plates were sealed with gas permeable sheets (ABgene, #AB-0718) and incubated at 25° C. At day 5 after RNAi treatment, the cells were pelleted by spinning at 2000 rpm for 2 min, medium was removed using a Biomek FX, plates were sealed and the cells were frozen at −80° C. until use. In each well, cell pellet was lyzed in 60 ul Renilla lysis buffer for 30 min. A aliquot of 20 ul cell lysate was used for CS activity assay, 20 ul for luciferase assay.

To measure CS activity for the whole genome screen and the confirmation screen, cells were lyzed in renilla lysis buffer (Promega E2820) and assays were performed in 384 well plates (Falcon, CAT#353962). Absorbance was measured every 46 second for 15 time points by plate reader (PerkinElmer, Envision 2100). CS activity was normalized by renillar luciferase activity. Vmax (SLOPE) and R² (RSQ) were calculated in Excel. Vmax (milliOD per minute) was only accepted if R² was more than 0.97. Otherwise, data points in the linear range were reselected manually and R² was recalculated until it was more than 0.97.

To measure renilla luciferase activity, the renilla luciferase assay system (Promega E2820) was used for the first 24 plates for the primary screen. Dual-Glo Luciferase Assay system (Promega E2980) was used for the rest of the plates for the primary screen and confirmation screen. Experiments were performed according to manufacturer's instructions. Luciferase activity was measured by Plate readers (PerkinElmer, Envision 2100).

To analyze primary screen data, for each well, there were two data values, the Vmax of CS activity and the renilla luciferase activity expressed as relative light unit (RLU). The ratio of Vmax/RLU was calculated for each well. The Vmax/RLU value was normalized by a two-dimensional normalization scheme after log transformation of the raw data. The first dimension normalization (1D) was done based on plate median of screened 384-well plates. Thus, the formula for calculating the 1D normalized value (denoted as x_{1D}) is x_{1D}=log(Vmax/RLU)/median(log(Vmax/RLU)). After 1D normalization, the second dimension normalization (2D) was done based on the median of a set of wells with each from a different plate, but sharing the same relative location on a plate, for example, A1 wells of all screened plates. The formula for calculating the 2D normalized value (denoted as x_{2D}) is x_{2D}=x_{1D}/median(x_{1D}). For x_{2D} of different plates and wells to be comparable, the normalized Z score (NZ) was calculated for every x_{2D} The formula used for calculating NZ_{2D} is: NZ_{2D}(x_{2D}−median(x_{2D}))/MAD(x_{2D}). MAD(x_{2D}) is Median Absolute Deviation of x and equals to 1.4826×median (|x_{2D}−median(x_{2D})|).

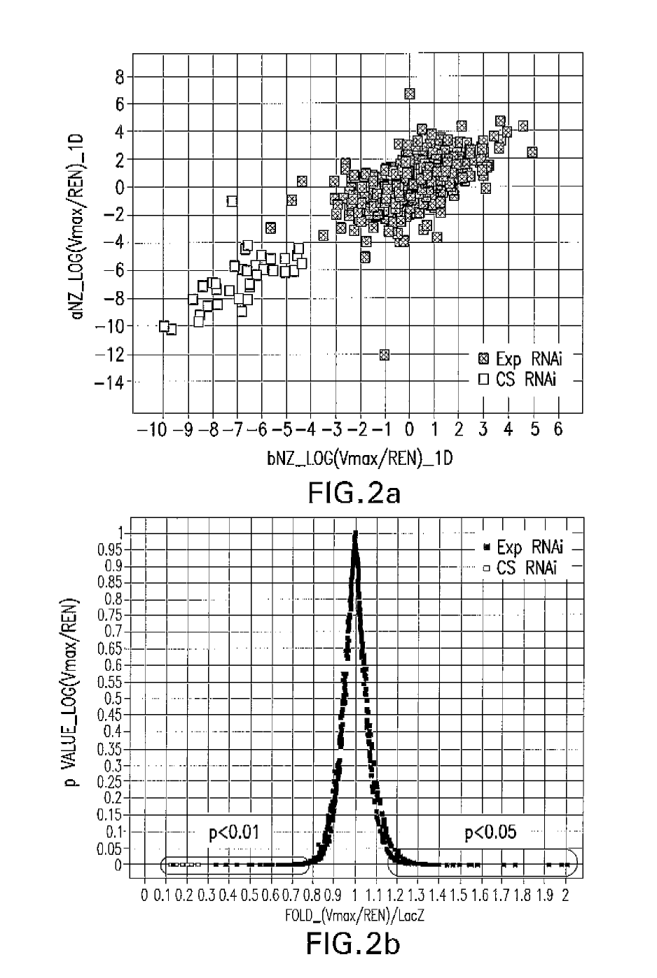

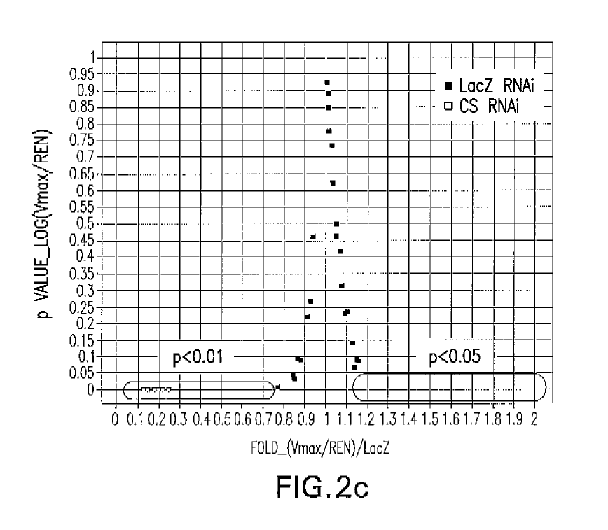

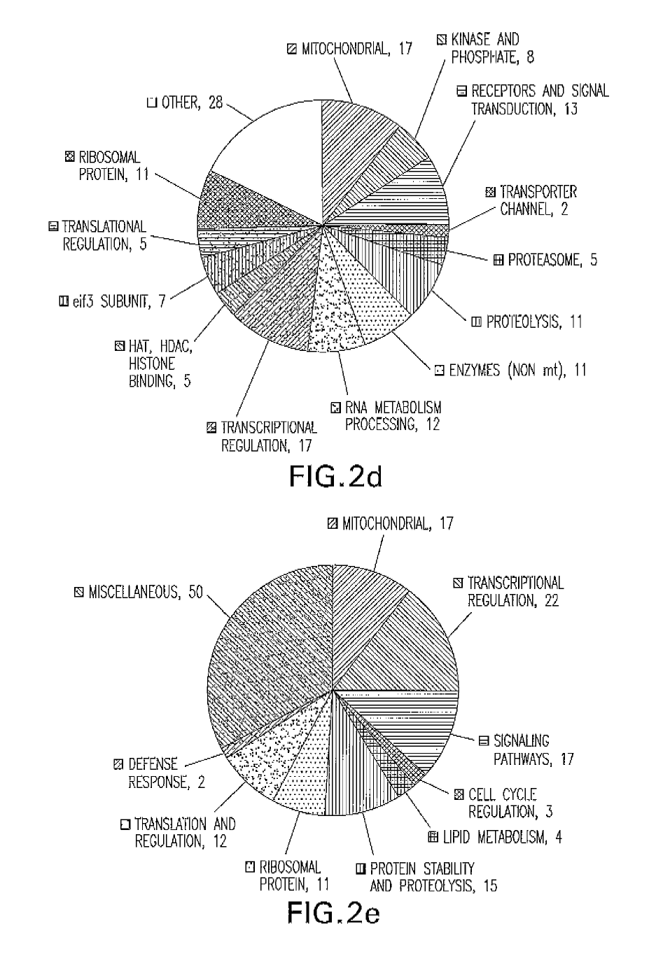

Primary hits were selected if NZ scores for both replicates were larger than 2 or smaller than −2. Additional hits with only one replica having NZ larger than 3 or smaller than −3 were also selected for re-testing. A total of 821 primary hits, approximately 6% of the collection, were selected from the primary screen (FIG. 2a). Among them, 573 of 821 genes that have human homologues were selected for confirmation.

To verify the hits, dsRNA of 573 hits were first cherry-picked into two 384-well plates, then aliquot into 6 replicate plates with 5 ul of dsRNA in each well at 50 ng/ul. Verification screen was performed the same way as whole genome screen except that the cells were harvested at Day 6 after RNAi.

To analyze confirmation screen data, p value (t-test) were calculated comparing to the LacZ RNAi controls. The average coefficient of variation (CV) of the CS assay was about 3.5%, on average our confirmation assay with six replicates was able to detect 5% or larger changes with p<0.05 (t-test). The power of our confirmation assay was greater than 0.8 when the change in mean CS activity was greater than 7% (t-test).

A total of 153 hits were verified after the confirmation step. Among them, 76 hits, whose dsRNA led to up-regulation of CS activity, were selected because their p-value were less than 0.05. The fold change of their CS activity over the LacZ control ranged from 1.15 to 2 fold (FIG. 2b). On the other hand, for those hits whose dsRNA led to down-regulation of CS activities, 77 hits had their p-value smaller than 0.01, their fold change of CS activities over the control range from 0.13 to 0.8 (FIG. 2b). To avoid false positives, p value of 0.01 was chosen as a cut-off since there were three LacZ RNAi controls having p values between 0.05 to 0.01 (FIG. 2c). Under our experimental conditions, changes of CS activity were observed as low as 15-20%, consistent with a previous report about the observable changes of CS activity. (Stump, C. S., et al. (2003) PNAS 100:7996).

### Example 2

### Analysis and Categorization of Hits

A total of 153 hits were identified from the whole genome RNAi screen (TABLE I). These genes were classified by their associated Gene Ontology annotation or InterPro protein domain (FIG. 2d). The hits cover a wide range of molecular functions, including mitochondrial associated function, kinases and phosphatases, receptors and signal transduction, proteosomal components and proteolysis, enzymes, RNA processing, transcriptional and translational regulators. Furthermore, to identify the potential pathways that regulate mitochondrial function, these hits were classified by their associated biological processes and pathways (FIG. 2e). The top three categories with hits associated with mitochondrial related functions (17 genes) (TABLE I), transcriptional regulation (22 genes) (TABLE I), and signaling pathway with (17 genes) (TABLE I). Moreover, for several protein complexes, multiple subunits were identified, indicating the validity of the screen. These include two subunits of pyruvate dehydrogenase, two subunits for α-ketoglutarate dehydrogenase, two subunits of ATP synthase, three subunits of NELF (negative elongation factor) complex and two subunits of TFIID (TABLE I).

Hits With Mitochondrial Function

11% (17/153) of the hits identified were from the RNAi screen encode proteins with mitochondrial functions (TABLE I). Compared to 2-3% genes encoding mitochondrial proteins in the whole genome, mitochondrial proteins were enriched by the screen, indicating the specificity of this RNAi screen. (Catalano, D., et al. (2006) BMC Bioinformatics 7:36) (Sardiello, M., et al. (2003) Nucleic Acids Research 31:322) These include E1β (CG11876) and E3 (CG7430) subunits of pyruvate dehydrogenase (PDH), and E1 (Nc73EF) and E3 (CG7430) subunits of α-ketoglutarate dehydrogenase. Interestingly, these two enzyme complexes have similar compositions and catalyze very similar chemical reactions. They both consist of E1, E2 and E3 subunits, and even share E3 subunits. These two enzyme complexes both utilize CoASH and NAD⁺ and yield CO₂ and NADH.

The reaction catalyzed by CS is the first rate-controlling steps in the TCA cycle, it is regulated by substrate availability and production inhibition. RNAi against PDH subunits resulted in an up-regulation of CS activity, which could be due to the compensation caused by the reduction of acetyl-CoA, a substrate for CS. Similarly, RNAi treatment of pyruvate carboxylase, resulted in an up-regulation of CS activity (TABLE I). Pyruvate carboxylase catalyzes the conversion of pyruvate to oxaloacetate which is another substrate for CS. On the other hand, RNAi against α-ketoglutarate dehydrogenase resulted in a reduction of CS activity. This could be due to product inhibition. α-ketoglutarate dehydrogenase catalyzes a biochemical reaction after CS in the TCA cycle. The reduction of α-ketoglutarate dehydrogenase will indirectly lead to an accumulation of the citrate, a product of CS, which will in turn inhibit CS activity. The identification of genes involved in both mechanisms of CS regulation, substrate availability and production inhibition, indicates that the screen is sensitive enough to uncover the enzymes that regulate CS activity through metabolic intermediates.

### Example 3

### Further Analysis of Hits in Transgenic Flies and Fly Mutants

To measure CS activity and Cytochrome C oxidase (COX) activity in flies, fly stocks were crossed to wild type strains. Sibling flies with and without mutations or transgenes were compared for their mitochondrial CS and/or COX activities. Since heterozygosity is generally expected to maximally reduce gene dosage by 50% at maximum, this in vivo validation method was expected to only uncover those genes whose rate-limiting dosage is above 50% of the wild type animals.

There are several advantages for using heterozygous animals, such as reducing the chance of triggering apoptosis and retrograde response, avoiding lethality associated with many homozygous mutations, and rapidly analyzing relatively large number of hits from the screen. Individual flies were homogenized, and an aliquot of lysate was used for CS activities and total protein concentration assay. Total protein concentration was used to normalize CS activities. Eight individual flies were analyzed for each fly genotype, providing eight biological replicates. Wild type strains were analyzed in parallel as additional controls for balancers or markers.

To Measure CS activity in single flies, reactions were carried out in 96 well plates (Costar, Cat. # 07-200-656). Single flies were lysed in extraction buffer containing 0.1% Triton X-100, 1 mM EDTA, and 20 mM HEPES (GIBCO, #15630-080, PH 7.2). Individual fly was homogenized in eppendorf tube with 250 ul extraction buffer, supernatant was transferred into the well of 96-well plate. Eight individual flies were analyzed for each fly genotype, providing eight biological replicates. 10 ul fly lysate were used for each reaction of CS assay. The total reaction volume was 100 ul, containing 0.1 mM DTNB (Sigma, Cat. # D8130), 0.3 mM acetyl-CoA (Roche, Cat. # 1585371) and 1 mM oxaloacetate (Sigma, Cat. # 04126) in 50 mM Tris pH 8.0 (USB, Cat. # 22638). Oxaloacetate was always added just before the measurements were taken. Absorbance was measured at 412 nm every 15 second for 10 min (at 25° C.) using the kinetic mode of spectrometer (Molecular Devices). The CS activity was normalized by total protein concentration (Bio-rad protein assay reagent).

To measure COX activity, Cytochrome C Oxidase Assay Kit (Sigma, cat #CYTO_COX1) was adapted into 96 well plate format. CytC solution was made at 5.4 mg/ml. For every 2.7 mg CytC, 10 ul DTT (0.1 mM) was added to reduce CytC until the ratio of absorbance 550 nm/565 nm is greater than 10. 50 ul lysate and 25 ul CytC were used for COX activity assays. Absorbance at 550 nm was acquired every 6 second for 1 mM. COX activity (Vmax) was calculated as the rate of reduction of absorbance with time. COX activity was normalized by total protein concentration. The same single fly lysate (250 ul extraction buffer per fly) was used for CS assay (10 ul), COX assay (50 ul) and for measuring total protein concentration (3 ul). Eight replicates (one fly per replicate) were used for each genotype.

Hits in Epigenetic Regulation

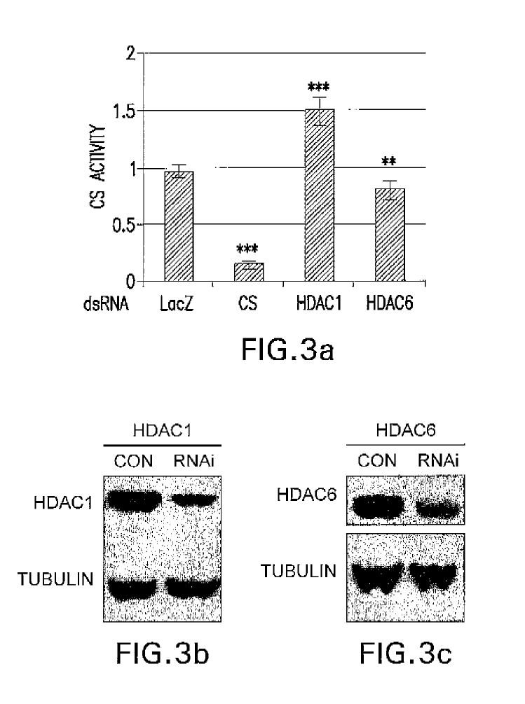

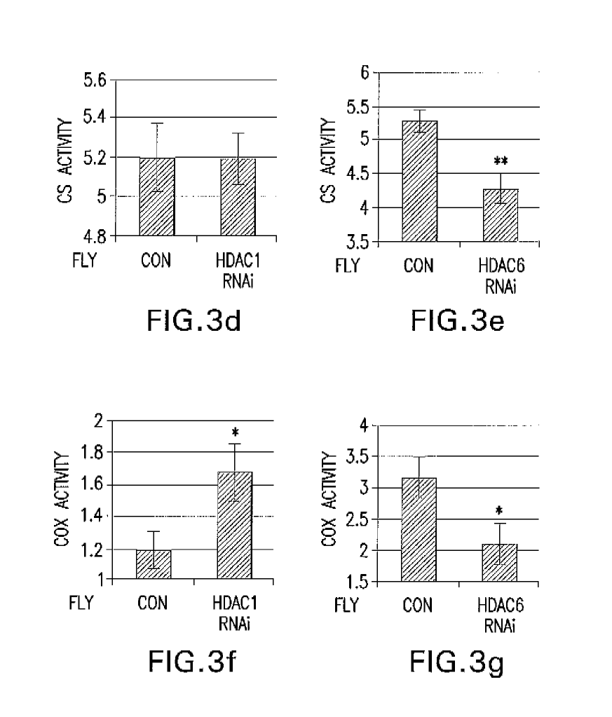

There were 14% (22/153) hits identified from the RNAi screen with their function in transcriptional regulation (TABLE I). Among them, HDAC1 was identified as a negative modulator and HDAC6 as a positive modulator of CS activity. Compared with LacZ RNAi controls, CS activity was increased by 50% in cells treated with HDAC1/Rpd3 RNAi, and was reduced by 20% with HDAC6 RNAi (FIG. 3a).

To find out in vivo significance of HDAC 1 and HDAC6 in regulating mitochondrial functions, the transgenic flies were generated with the HDAC1 or HDAC6 RNAi constructs under the GAL4-UAS induction. These transgenic flies were lethal prior to adult stage with a global induction of RNAi under the tubulin-GAL4 driver, but viable and fertile with a muscle specific induction of RNAi under the mef-Gal4 driver. HDAC1 and HDAC6 protein levels were highly reduced in these transgenic flies, indicating the efficiency of RNAi in knocking-down the target genes (FIG. 3b, c). CS activity was then measured in adult HDAC RNAi flies under the induction of mef-GAL4 driver. In order to find out whether HDACs affect other mitochondrial function, the activity of cytochrome c oxidase (COX), a marker of total oxidative phosphorylation capacity, was also analyzed in parallel.

In HDAC1 RNAi transgenic flies, COX activity was increased 41% compared to their sibling control flies, indicating that HDAC1 negatively modulate mitochondrial COX activity in vivo (FIG. 3d, e). However, a change in CS activity was not observed in HDAC1 RNAi transgenic flies. This might be due to the limitation of muscle specific induction of RNAi. Small changes of CS activity in muscles might be underestimated when assayed with whole fly lysates.

The observed different in vivo effect of HDAC1 on CS and COX activities may be explained by the intrinsic difference between the CS and COX enzyme complexes. The COX complex contains as many as 13 subunits encoded by both nuclear genome and mitochondrial genome, while CS acts as homodimer. It is conceivable that the subunits of COX complex work synergistically and small perturbations on two or more subunits caused by HDAC1 RNAi may lead to a strong effect on COX activity, while the small effect on CS protein by HDAC1 RNAi may not translate to large non-linear effect on CS activity that is easily detectable. Indeed, HDAC1 seems to affect quite a number of mitochondrial proteins. Microarray studies have been performed in Drosophila cells treated with Sin3A (cofactor of HDAC1) RNAi, and a significant number of genes involved in mitochondrial processes found to be affected. (Pile, L. A., et al. (2003) JBC 278:37840) Most of these genes were repressed by Sin3A.

In HDAC6 RNAi transgenic flies, CS activity was reduced 19% and COX activity was reduced 34% compared to their sibling controls, indicating that HDAC6 positively modulate mitochondrial functions in vivo (FIG. 3f, h). HDAC6 has not been reported previously to have a mitochondrial function, however, several HDAC6 substrates are known to play a role in mitochondrial function. HDAC6 functions as an Hsp90 deacetylase and is required for hsp90-dependent maturation of glucocoticoid receptor (GR) (Murphy, P. J., et al. (2005) Journal of Biological Chemistry 280:33792) (Kovacs, J. J., et al. (2005) Molecular Cell 18:601). GR was shown to localize in mitochondria and stimulate mitochondrial biogenesis and function, including COX activity. (Psarra, A. M., et al. (2005) International Journal of Biochemistry & Cell Biology 37: 2544) (Scheller, K., et al. (2003) Experimental Physiology 88:129) (Weber, K., et al. (2002) Endocrinology 143:177) HDAC6 also deacetylates α-tubulin in vivo and in vitro (without affecting tubulin protein level), and destabilizes the microtubular network. (Kovacs, J. J., et al. (2005) Molecular Cell 18:601) (Zhang, Y., et al. (2003) EMBO Journal 22:1168) (Matsuyama, A., et al. (2002) EMBO Journal 21:6820) (Hubbert, C., et al. (2002) Nature 417:455) (Haggarty, S. J., et al. (2003) PNAS 100:4389) (Iwata, A., et al. (2005) JBC 280:40282)

Mitochondria are known to require an intact microtubular network to function. (Appaix, F., et al. (2003) Experimental Physiology 88:175) The HDAC6 mitochondrial function seen is consistent with previous studies about the roles of HDAC6 substrates in mitochondria.

The present study provides in vivo evidence for HDAC1 and HDAC6 in mitochondrial function. It was shown previously that HDAC5 negatively regulates mitochondrial biogenesis in mammalian cells. (Czubryt, M. P., et al. (2003) PNAS 100: 1711) HDAC7 has been reported to have mitochondrial localization. (Bakin, R. E., et al. (2004) JBC 279:51218) Significant changes in CS activity in cells treated with RNAi of HDACs other than HDAC1 and HDAC6 were not observed.

Hits in Signaling Pathways

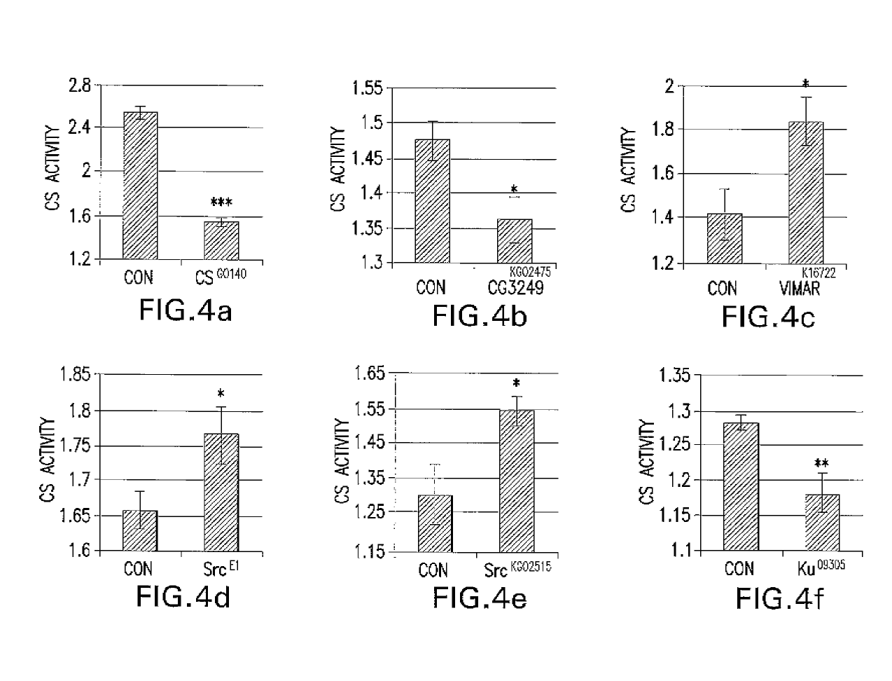

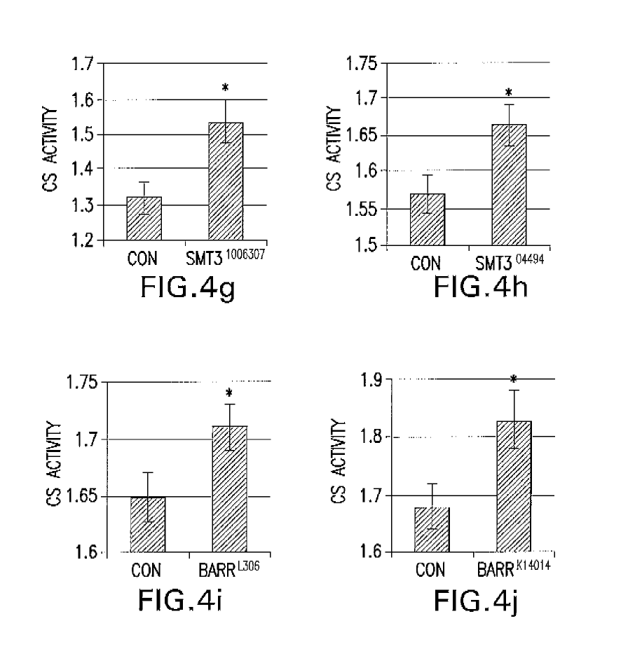

There were 11% (17 out of 153) hits identified for their function in signaling pathways (TABLE I). The study for the effect of signaling pathways on mitochondrial function is limited in model organisms. Taking advantage of the publicly available large collection of fly mutants, a number of hits were further analyzed for their mitochondrial functions in mutation flies. CS heterozygous mutants was first tested as a positive control. CS activity was reduced 39% in heterozygous CS mutants compared to their wild type siblings (FIG. 4a). Twenty three hits were then analyzed with their mutations in heterozygous backgrounds. Six of them were shown to have significant effects on CS activity, including CG3249, vimar, Src42A, klumpfuss, smt3 and barren (FIG. 4).

Two genes in the PKA (cAMP-dependent protein kinase) pathway, CG3249 and PKA, were identified in the screen (TABLE I). CS activity was reduced 8% in CG3249 heterozygous mutants. CG3249 encodes a homology of the A-kinase anchor protein (AKAP). A role for AKAP in regulating mitochondria function has been reported previously. (Horbinski, C., et al. (2005) Free Radical Biology & Medicine 38:2) (Livigni, A., et al. (2006) Molecular Biology of the Cell 17:263) It was shown that AKAP anchors PKA to mitochondria, increases PKA-dependent phosphorylation of the proapoptotic protein BAD, and enhances cell survival. (Affaitati, A., et al. (2003) JBC 278:4286) (Harada, H., et al. (1999) Molecular Cell 3:413) AKAP was shown to target the PTPD1/Src complex to the mitochondria to enhance Src-dependent tyrosine phosphorylation of mitochondrial substrate proteins (Livigni 2006) (Cardone, L., et al. (2004) Molecular & Cellular Biology 24:4613). CG3249 heterozygous mutants were shown to have reduced CS activity (FIG. 4b), supporting a positive role for AKAP in regulating mitochondrial function.

Three genes in the small GTPase mediated signaling pathway were identified (TABLE I). A significant 30% increase in CS activity was observed in vimar heterozygous mutants (FIG. 4c). The human homolog of vimar is RAP1GDS1, a guanine exchange factor (GEF) that activates RAP1. RAP1 is a small G protein involved in the regulation of the actin cytoskeleton. (Bos, J. L. (2005) Current Opinion in Cell Biology 17:123) A number of GEFs have been identified for RAP1. (Bos 2005) One type of RAP1-GEFs, Epac, was observed to localize to mitochondria and activate RAP1 by direct binding of cAMP in a PKA independent manner. (Wang, Z., et al. (2006) Molecular & Cellular Biology 26:213) (Qiao, J., et al. (2002) JBC 277:26581) Here, another RAP1GEF was identified as modulating mitochondrial function, supporting a mitochondrial role for RAP1.

Src42A, a tyrosone kinase, was also analyzed in fly mutants for its role in mitochondrial function. CS activities were increased 7% and 18% in two different hetereozygous Src42A mutants (FIG. 4d, e). Src42A encodes the closest relative of vertebrate Src in Drosophila. While nine members of Src kinase were found in vertebrates, only two Src kinases, Src42A and Src64, were identified in Drosophila. (Takahashi, F., et al. (1996) Genes & Development 10:1645) (Simon, M. A., et al. (1985) Cell 42:831) They have redundant functions in regulating JUN kinase (JNK) activity in Drosophila. (Tateno, M., et al. (2000) Science 287:324) (Takahashi, M., et al. (2005) Development 132:2547) Src42A was also shown to negatively regulate RTK (receptor tyrosine kinase) signaling. (Lu, X., et al. (1999) Developmental Biology 208:233) The first evidence for Src kinase in mitochondrial localization was found in rat brain. (Salvi, M., et al. (2002) Biochimica et Biophysica Acta 1589:181) Another study showed that c-Src phosphorylates and actives cytochrome c oxidase. (Miyazaki, T., et al. (2003) Journal of Cell Biology 160:709) (Miyazaki, T., et al (2004) JBC 279:17660) The homolog of c-Src in Drosophila is Src64. (Simon, M. A., et al. (1983) Nature 302:837) Under our experimental condition, Src42A appears to negatively interfere with CS activity, supporting a role of Src kinase in regulating mitochondrial function.

Two genes in apoptosis pathway, klumpfuss and thread/DIAP1, were identified from the screen. CS activity was reduced 8% in klumpfuss heterozygous mutants (FIG. 4f). The function of klumpfuss was shown to promote programmed cell death by down-regulation of cell survival signals mediated by EGFR (Epiermal Growth factor receptor)/dRAS1 signaling pathway. (Rusconi, J. C., et al. (2004) Mechanisms of Development 121:537) The data described herein suggest a cross talk between apoptosis pathway and mitochondrial functions.

Hits in Other Biological Processes

CS activities were significantly increased, 17% and 6%, in two different smt3 heterozygous mutants (FIG. 4g, h). smt3 encodes a Drosophila member of SUMO (small ubiquitin-related modifier) protein. There are four SUMO isoforms (SUMO1-4) found in mammals and one SUMO in Drosophila. SUMO modification appears to have diverse targets and play important roles in protein stability, protein targeting and other processes. (Dohmen, R. J. et al. (2004) Biochimica et Biophysica Acta 1695:113) SUMO proteins conjugate to other proteins via a very similar mechanism with that of ubiquitin system. SUMO is first activated by SUMO-activating enzyme (E1), then transferred to a conjugating enzyme (E2), after conjunction with the substrate recognizing SUMO ligases (E3), E2 conjugates SUMO to a variety of substrate proteins. (Dohmen 2004)

The first mitochondrial target identified for SUMO is DRP1, which functions in mitochondrial fission, and its Drosophila ortholog shibire was also identified in our screen (TABLE I). DRP1 was shown to colocalize with SUMO1 and to be sumolated in mammalian cells. (Harder, Z., et al. (2004) Current Biology 14:340) Here, CS activity was increased in smt3 heterozygous mutants, providing additional support for sumoylation in mitochondrial protein modification.

Three hits in cell cycle regulation were identified from the RNAi screen and function in mitotic regulation (TABLE I). CS activities were increased 4% and 9% in two different barren heterozygous mutants (FIG. 4i, j). barren encodes a subunit of condensin, a complex required for chromosome condensation. (Legagneux, V., et al. (2004) Biology of the Cell 96:201) (Hirano, T., et al. (1997) Cell 89:511) barren was first characterized in Drosophila, and homozygous barren mutants were embryonic lethal with a severe loss of PNS neurons. (Kania, A., et al. (1995) Genetics 139:1663) (Bhat, M. A., et al. (1996) Cell 87:1103) It is possible that mitosis signals to mitochondria to coordinate energy production with growth. It was shown that low glucose and ATP levels lead to cell cycle arrest and promotes cell survival via p53 pathway. (Jones, R. G., et al. (2005) Molecular Cell 18:283) It was proposed that a low-energy cell-cycle checkpoint monitors the metabolic activity of the mitochondria before committing to another round of cell division. (McBride, H. M., et al. (2006) Current Biology 16:R551)

1. A method to treat, prevent or ameliorate a condition associated with mitochondrial dysfunction, comprising administering to a subject in need thereof an effective amount of a modulator of a protein selected from the group consisting of the proteins disclosed in TABLE I.

2. The method of claim 1, wherein said condition is a neurodegenerative disease.

3. The method of claim 1, wherein said condition is diabetes.

4. The method of claim 1, wherein said condition is an age-related disorder.

5. The method of claim 1, wherein said condition is a cancer.

6. The method of claim 1, wherein said modulator inhibits a biological activity of said protein in said subject.

7. The method of claim 6, wherein said modulator comprises one or more antibodies or fragments thereof that bind said protein, wherein said one or more antibodies or fragments thereof inhibit a biological activity of said protein in said subject.

8. The method of claim 1, wherein said modulator enhances a biological activity of said protein in said subject.

9. The method of claim 1, wherein said modulator inhibits expression of a gene encoding said protein in said subject.

10. The method of claim 9, wherein said modulator comprises one or more substances selected from the group consisting of an antisense oligonucleotide, a triple-helix DNA, a ribozyme, an RNA aptamer, a siRNA, a double-stranded RNA, and a single-stranded RNA.

11. The method of claim 1, wherein said modulator enhances expression of a gene encoding said protein in said subject.

12. A method to treat, prevent or ameliorate a condition associated with mitochondrial dysfunction comprising administering to a subject in need thereof a pharmaceutical composition comprising an effective amount of a modulator of a protein selected from the group consisting of the proteins disclosed in TABLE I.

13. The method of claim 12, wherein said condition is a neurodegenerative disease.

14. The method of claim 12, wherein said condition is diabetes.

15. The method of claim 12, wherein said condition is an age-related disorder.

16. The method of claim 12, wherein said condition is a cancer.

17. The method of claim 12, wherein said modulator inhibits a biological activity of said protein in said subject.

18. The method of claim 12, wherein said modulator comprises one or more antibodies that bind said protein, or fragments thereof, wherein said one or more antibodies or fragments thereof inhibit a biological activity of said protein in said subject.

19. The method of claim 18, wherein said modulator enhances a biological activity of said protein in said subject.

20. The method of claim 18, wherein said modulator inhibits expression of a gene encoding said protein in said subject.

21. The method of claim 20, wherein said modulator comprises one or more substances selected from the group consisting of an antisense oligonucleotide, a triple-helix DNA, a ribozyme, an RNA aptamer, a siRNA, a double-stranded RNA, and a single-stranded RNA.

22. The method of claim 12, wherein said modulator enhances expression of a gene encoding said protein in said subject.

23.-47. (canceled)

48. A method to diagnose a subject suffering from a condition associated with mitochondrial dysfunction that may be a suitable candidate for treatment with a modulator of a protein selected from the group consisting of proteins disclosed in TABLE I, comprising assaying the mRNA level whose translation provides any one or more of said proteins in a biological sample from said subject wherein a subject with an altered mRNA level compared to a control is a suitable candidate for modulator treatment.

49. A method to diagnose a subject suffering from a condition associated with mitochondrial dysfunction who may be a suitable candidates for treatment with a modulator of a protein selected from the group consisting of proteins disclosed in TABLE I, comprising detecting the level of any one or more of said proteins in a biological sample from said subject wherein subjects with altered levels compared to a control is a suitable candidate for modulator treatment.

50. A method to treat, prevent or ameliorate a condition associated with mitochondrial dysfunction comprising:
(a) assaying for the level of an mRNA encoding a protein selected from the group consisting of the proteins disclosed in TABLE I in a biological sample from a subject; and
(b) administering to a subject with altered levels of mRNA of said protein compared to controls a modulator to said protein in an amount sufficient to treat, prevent or ameliorate the pathological effect of said condition.

51. The method of claim 50, wherein said condition is a neurodegenerative disease.

52. The method of claim 50, wherein said condition is diabetes.

53. The method of claim 50, wherein said condition is an age-related disorder.

54. The method of claim 50, wherein said condition is a cancer.

55. The method of claim 50, wherein said modulator enhances the gene expression of said protein.

56. The method of claim 50, wherein said modulator inhibits the gene expression of said protein.

57. A method to treat, prevent or ameliorate a condition associated with mitochondrial dysfunction comprising:
(a) assaying for the level of a protein selected from the group consisting of the proteins disclosed in TABLE I in a biological sample from a subject; and
(b) administering to a subject with altered levels of said protein compared to a control a modulator of said protein in an amount sufficient to treat, prevent or ameliorate the pathological effects of said condition.

58. The method of claim 57, wherein said condition is a neurodegenerative disease.

59. The method of claim 57, wherein said condition is diabetes.

60. The method of claim 57, wherein said condition is an age-related disorder.

61. The method of claim 57, wherein said condition is a cancer.

62. The method of claim 57, wherein said modulator enhances a biological activity of said protein.

63. The method of claim 57, wherein said modulator inhibits a biological activity of said protein.

64. A diagnostic kit for detecting mRNA levels of a protein selected from the group consisting of the proteins disclosed in TABLE I in a biological sample, said kit comprising:
(a) a polynucleotide encoding a polypeptide set forth in TABLE I or a fragment thereof;
(b) a nucleotide sequence complementary to that of (a);
(c) a polypeptide of TABLE I of the present invention encoded by the polynucleotide of (a);
(d) an antibody to the polypeptide of (c);
(e) an RNAi sequence complementary to that of (a),
wherein components (a), (b), (c), (d) or (e) may comprise a substantial component.

65. A diagnostic kit for detecting levels of a protein selected from the group consisting of the proteins disclosed in TABLE I in a biological sample, said kit comprising:
(a) a polynucleotide of a polypeptide set forth in TABLE I or a fragment thereof;
(b) a nucleotide sequence complementary to that of (a);
(c) a polypeptide of TABLE I of the present invention encoded by the polynucleotide of (a);
(d) an antibody to the polypeptide of (c);
(e) an RNAi sequence complementary to that of (a),
wherein components (a), (b), (c), (d) or (e) may comprise a substantial component.
